(19) United States
(12) Patent Application Publication
(10) Pub. No.: US 2026/0077129 A1
(43) Pub. Date: Mar. 19, 2026

(54) READY TO USE DETECTORS

(71) Applicant: JANSSEN BIOTECH, INC., Horsham, PA (US)

(72) Inventors: EMMA LOUISE HUBERT, SAN JOSE, CA (US); JINGLI WANG, SAN JOSE, CA (US); MONICA A. KAPIL, SAN JOSE, CA (US); BRANDON MCKENZIE, WEST CHESTER, PA (US)

(21) Appl. No.: 19/109,273

(22) PCT Filed: Sep. 26, 2023
(86) PCT No.: PCT/IB2023/059480

§ 371 Date: Mar. 6, 2025

Related U.S. Application Data
(60) Provisional application No. 63/377,344, filed on Sep. 27, 2022.

(51) Int. Cl. A61M 5/20; A61M 5/31

(52) U.S. Cl. CPC A61M 5/20; A61M 2005/2006; A61M 2005/3125; A61M 2205/3368

(57) ABSTRACT

Provided herein are systems and methods for detecting the temperature of and indicating if a pharmaceutical product is ready to be used after a specified amount of time has elapsed. The detectors provided herein are configured to be attached to the pharmaceutical product that contains an active pharmaceutical ingredient (API) therein, and comprise a temperature sensor configured to detect a temperature of the pharmaceutical product; at least one timer configured to start measuring a passage of time if the temperature of the pharmaceutical product detected by the temperature sensor reaches or exceeds a breach temperature; and at least one indicator configured to generate, if a specified amount of time has elapsed on the at least one timer, an indication that the pharmaceutical product is ready to be used.

### FIELD

The present disclosure generally relates to detectors for pharmaceutical products, and in particular, to detectors that determine and indicate when a pharmaceutical product is ready to be used.

### BACKGROUND

Pharmaceutical products are distributed with instructions for use (IFUs) on a label and/or package insert which informs the user how and when to use the pharmaceutical product. For example, the pharmaceutical product can comprise an active pharmaceutical ingredient (API) which requires storage in a cool environment (e.g., a refrigerator, freezer, etc.) up until consuming the API to maintain the effectiveness, sterility, and/or physical form of the API. The duration of time that an API can be kept outside of the controlled cooling environment varies between APIs. The IFU informs the user of a duration of time that the API must sit in an ambient environment (e.g., outside of the refrigerator) prior to using the API, as well as the maximum amount of time the API can be kept outside of the refrigerator before expiring.

Drug delivery performance is highly dependent on temperature-sensitive formulation properties of the API, such as viscosity and density. APIs can be injected (e.g., manually by a user or using an autoinjector), ingested/consumed orally, or inhaled, for example. When injecting a drug through a needle, the injection force (e.g., for manual injection) and injection time (e.g., for mechanical assist delivery such as autoinjectors) are highly dependent on the formulation temperature. API temperature is inversely proportional to viscosity, injection time, and injection force, such that lower temperatures cause higher viscosity, longer injection time, and require larger injection forces. Thus, it is required that the drug product (e.g., API) rests outside of the controlled cooling environment in an ambient environment for a duration of time to decrease the viscosity, increase the temperature, therefore reducing the force required to inject the API (either manually or through use of an autoinjector) and the time of injection.

### SUMMARY

Provided herein are systems, devices, and methods for determining and indicating when a pharmaceutical product is ready to be used. The detectors described herein may detect temperatures of the pharmaceutical product, measure a passage of time once a breach temperature is reached or exceeded, and indicate, after a specified amount of time has elapsed, if the pharmaceutical product is ready to be used.

As mentioned above, pharmaceutical products are traditionally distributed with instructions for use (IFUs) on a product label and/or package insert that informs the user how/when to use a pharmaceutical product. For example, a pharmaceutical product may comprise an active pharmaceutical ingredient (API) that is required to be stored in a cool, controlled environment (e.g., refrigerator, freezer, etc.) up until the time of consumption to maintain the stability of the API. The IFU informs the user of a minimum and maximum duration of time that the pharmaceutical product comprising the API must rest at an ambient temperature (e.g., room temperature) prior to using the pharmaceutical product. The API may be required to rest at room temperature at least because drug delivery performance (e.g., injection time and/or injection force) may be highly dependent on the viscosity of the fluid, wherein viscosity is inversely related to the temperature of the API. Thus, at cooler temperatures, the viscosity of the API is higher, which leads to prolonged injection times and greater required injection forces that may exceed a user's and/or autoinjector's capacity. Despite informing the user of a wait time in an IFU, as described in greater detail below, the wait times observed by a user prior to using a pharmaceutical product are often inefficient, inaccurate, and ignored by the user.

For example, users may not read the IFU materials, and rather will crudely estimate a duration of time that the pharmaceutical product must rest outside of the controlled cooling environment prior to using the product. In some embodiments, a user may not abide by the IFU or medical professional instructions at all and rather may attempt to use (e.g., inject) the API of the pharmaceutical product without waiting any duration of time after the pharmaceutical product is removed from the controlled cooling environment. In some instances, a user may forget about the pharmaceutical product after removing it from the controlled cooling environment and leave the product in an ambient environment for a duration of time that exceeds the maximum duration of time for the product to be left in the ambient environment, thus causing the API to expire.

Additionally, the duration of time that the pharmaceutical product may be required to rest outside of the cooling environment may vary based on the temperature of the environment at which the pharmaceutical product is placed in. For example, if the pharmaceutical product is placed in an environment warmer than ambient temperature, it may require less time to reach an appropriate temperature for use. On the other hand, if the pharmaceutical product is placed in an environment cooler than ambient temperature (yet still above the temperature range of the controlled cooling environment), it may require more time to reach an appropriate temperature for use. In some instances, the pharmaceutical product may never reach a desired use temperature or may require an excessive amount of time to reach the desired use temperature. Additionally, users may use multiple different pharmaceutical products each day, and each product may require a different duration of time to warm up to an appropriate use temperature; or the appropriate use temperature may be different for each product. Thus, a user may incorrectly correlate a wait time with a given pharmaceutical product and incorrectly use their pharmaceutical products.

Incorrectly using an API can lead to many issues for the user. For example, in the instance the API is injected in the user, the injection time may be prolonged if the API has not reached an appropriate temperature for use. As mentioned above, this is caused by a higher viscosity of the API, which is inversely related to the temperature of the API. In some instances, the injector may fail to fully inject (or inject at all) the API if the API is not at the appropriate use temperature. For example, the cool temperature and high viscosity may require a large injection force that exceeds the capabilities of the user, or, in the instance the API is injected with an autoinjector, exceeds the maximum spring force that may be exerted by the spring of the autoinjector. Furthermore, injecting an API that has not reached the appropriate temperature may cause user discomfort and pain at least at the injection site. The pain may be caused at least from the cool temperature of the API and/or the high viscosity of the fluid, which may not allow the fluid to disperse once injected. Additionally, in the instance the pharmaceutical product is left out of the controlled cooling environment beyond the informed maximum duration of time (e.g., the user forgot about the product after removing it from the cooling environment), the pharmaceutical product may lose its efficacy, and worse, expire. For example, the sterility of the pharmaceutical product may be compromised, and the physical form of the API may be diminished thus causing the pharmaceutical product to be unsuitable for use.

Provided herein are detectors configured to be attached to a pharmaceutical product. The detectors are configured to automatically detect the temperature of the pharmaceutical product, measure one or more passages of time once one or more breach temperatures are reached or exceeded, and indicate, after a specified amount of time, if the pharmaceutical product is ready to be used. The detectors may alleviate any risk of user confusion and more efficiently inform the user when the pharmaceutical product is ready to be used by tracking both time and temperature. Pharmaceutical products may include, for example, injectors (e.g., autoinjectors, syringes, manual injectors, etc.) and containers (e.g., vials, bottles, cartridges, etc.) that contain or are configured to contain an active pharmaceutical ingredient (API) (e.g., pharmaceutical, medication, antibiotic, vaccine, drug, etc.).

In some embodiments, a detector configured to be attached to a pharmaceutical product that contains an active pharmaceutical ingredient (API) therein is provided, the detector comprising: a temperature sensor configured to detect a temperature of the pharmaceutical product; at least one timer configured to start measuring a passage of time if the temperature of the pharmaceutical product detected by the temperature sensor reaches or exceeds a breach temperature; and at least one indicator configured to generate, if a specified amount of time has elapsed on the at least one timer, an indication that the pharmaceutical product is ready to be used.

In some embodiments, the specified amount of time is selected to correspond to when the active pharmaceutical ingredient (API) within the pharmaceutical product will be ready to be used.

In some embodiments, the specified amount of time is selected to correspond to an amount of time in which the active pharmaceutical ingredient (API) contained within the pharmaceutical product will, when disposed in an environment that is within a standard room temperature range, reach a desired temperature.

In some embodiments, the breach temperature corresponds with a temperature outside of a standard refrigeration temperature range.

In some embodiments, a pharmaceutical product is provided, comprising: a container configured to contain an active pharmaceutical ingredient (API); and a detector disposed adjacent to the container.

In some embodiments, the container comprises an injector configured to contain the active pharmaceutical ingredient (API).

In some embodiments, the injector comprises an autoinjector.

In some embodiments, the temperature sensor determines a temperature of the active pharmaceutical ingredient (API) based at least in part on a temperature of the container. In some embodiments, the detector is removably attached to the container.

In some embodiments, the detector comprises a user-controlled activator configured to activate the temperature sensor.

In some embodiments, the at least one timer is configured to start measuring a second passage of time if the temperature of the pharmaceutical product detected by the temperature sensor reaches or exceeds a second breach temperature.

In some embodiments, the second breach temperature is greater than the breach temperature.

In some embodiments, a first timer of the at least one timer is configured to measure the passage of time if the breach temperature is detected, and a second timer of the at least one timer is configured to measure the second passage of time if the second breach temperature is detected.

In some embodiments, the passage of time measured by the at least one timer is a longer duration of time than the second passage of time measured by the at least one timer.

In some embodiments, the at least one indicator is configured to generate, if a second specified amount of time has elapsed on the at least one timer, the indication that the pharmaceutical product is ready to be used.

In some embodiments, the at least one indicator comprises at least one of an audio indicator and/or a visual indicator.

In some embodiments, the visual indicator comprises a first illuminator configured to be activated when the pharmaceutical product is ready to be used.

In some embodiments, the visual indicator comprises a second illuminator configured to be activated if the pharmaceutical product is not ready to be used.

In some embodiments, the audio indicator is configured to produce a sound if the pharmaceutical product is ready to be used.

In some embodiments, the indicator comprises a graphical user interface (GUI) on a mobile device configured to indicate when the pharmaceutical product is ready to be used.

In some embodiments, the mobile device is communicatively coupled to one or more of the temperature sensor and timer.

In some embodiments, the graphical user interface (GUI) is configured to display an expected duration for the injection based at least in part on the detected temperature and the pharmaceutical product.

In some embodiments, the detector comprises a fluid configured to travel through the temperature sensor and the at least one timer.

In some embodiments, the detector comprises a user-controlled activator configured to cause, if a user-controlled activator is activated, the fluid to travel from a reservoir associated with the user-controlled activator and to the temperature sensor.

In some embodiments, the temperature sensor comprises a window configured to indicate that the detector is active.

In some embodiments, the fluid is configured to travel, if the pharmaceutical product reaches or exceeds the breach temperature, from the temperature sensor and through the at least one timer.

In some embodiments, the fluid is configured to travel through the at least one timer and to the at least one indicator for the specified amount of time.

In some embodiments, the specified amount of time corresponds to when the active pharmaceutical ingredient (API) within the pharmaceutical product will be ready to be used.

In some embodiments, the fluid is configured to travel, if the pharmaceutical product reaches or exceeds a second breach temperature, from the temperature sensor and through a second timer of the at least one timer.

In some embodiments, the fluid is configured to travel through the second timer and to the at least one indicator for a second specified amount of time.

In some embodiments, the second breach temperature is greater than the breach temperature, and wherein the second specified amount of time is higher than the specified amount of time.

In some embodiments, a method of indicating that a pharmaceutical product is ready to be used is provided, the method comprising: detecting, with a temperature sensor, a temperature of the pharmaceutical product; measuring, with at least one timer, a passage of time if the temperature of the pharmaceutical product detected by the temperature sensor reaches or exceeds a breach temperature; and generating, with at least one indicator and if a specified amount of time has elapsed on the at least one timer, an indication that the pharmaceutical product is ready to be used.

In some embodiments, the specified amount of time is selected to correspond to when the active pharmaceutical ingredient (API) within the pharmaceutical product will be ready to be used.

In some embodiments, the specified amount of time is selected to correspond to an amount of time in which the active pharmaceutical ingredient (API) contained within the pharmaceutical product will, when disposed in an environment that is within a standard room temperature range, reach a desired temperature.

In some embodiments, the breach temperature corresponds with a temperature outside of a standard refrigeration temperature range.

In some embodiments, the method comprises activating the temperature sensor with a user-controlled activator.

In some embodiments, the method comprises measuring, with the at least one timer, a second passage of time if the temperature of the pharmaceutical product detected by the temperature sensor reaches or exceeds a second breach temperature.

In some embodiments, the second breach temperature is greater than the breach temperature.

In some embodiments, a first timer of the at least one timer is configured to measure the passage of time if the breach temperature is detected, and a second timer of the at least one timer is configured to measure the second passage of time if the second breach temperature is detected.

In some embodiments, the passage of time measured by the at least one timer is a longer duration of time than the second passage of time measured by the at least one timer.

In some embodiments, the method comprises generating, with the at least one indicator and if a second specified amount of time has elapsed on the at least one timer, the indication that the pharmaceutical product is ready to be used.

In some embodiments, the at least one indicator comprises at least one of an audio indicator and/or a visual indicator.

In some embodiments, the method comprises activating the at least one visual indicator if the pharmaceutical product is ready to be used.

In some embodiments, the method comprises activating a second visual indicator of the at least one visual indicator if the pharmaceutical product is not ready to be used.

In some embodiments, the method comprises activating the audio indicator to produce a sound if the pharmaceutical product is ready to be used.

In some embodiments, the method comprises indicating, on a graphical user interface (GUI) on a mobile device, that the pharmaceutical product is ready to be used.

In some embodiments, the mobile device is communicatively coupled to one or more of the temperature sensor and timer.

In some embodiments, the method comprises displaying, on the graphical user interface (GUI), an expected duration for the injection based at least in part on the detected temperature and the pharmaceutical product.

In some embodiments, the method comprises a fluid configured to travel through the temperature sensor and the at least one timer.

In some embodiments, the method comprises causing, with a user-controlled activator, the fluid to travel from a reservoir associated with the user-controlled activator and to the temperature sensor.

In some embodiments, the fluid travels from the temperature sensor and through the at least one timer if the pharmaceutical product reaches or exceeds the breach temperature.

In some embodiments, the fluid travels through the at least one timer and to the at least one indicator for the specified amount of time.

In some embodiments, the specified amount of time corresponds to when the active pharmaceutical ingredient (API) within the pharmaceutical product will be ready to be used.

In some embodiments, the fluid travels from the temperature sensor and through a second timer of the at least one timer if the pharmaceutical product reaches or exceeds a second breach temperature.

In some embodiments, the fluid travels through the second timer and to the at least one indicator for a second specified amount of time.

In some embodiments, the second breach temperature is greater than the breach temperature, and wherein the second specified amount of time is higher than the specified amount of time.

In some embodiments, a detector configured to be attached to a pharmaceutical product that contains an active pharmaceutical ingredient (API) therein is provided, the detector comprising: a temperature sensor configured to detect temperatures of the pharmaceutical product; at least one processor configured to calculate changes between the detected temperatures over time; and at least one indicator configured to generate, if a change of the calculated changes between the detected temperatures over time meets or falls below a predefined threshold, an indication that the pharmaceutical product is ready to be used.

In some embodiments, the predefined threshold is selected to correspond to when the active pharmaceutical ingredient (API) within the pharmaceutical product will be ready to be used.

In some embodiments, calculating changes between the detected temperatures over time comprises determining a difference between a first temperature and a second temperature, the first and second temperature detected a predefined amount of time apart.

In some embodiments, the at least one processor is configured to generate a ratio of the difference between the first and second temperature over the predefined amount of time apart and compare the ratio to the predefined threshold.

In some embodiments, a method of indicating that a pharmaceutical product is ready to be used is provided, the method comprising: detecting, with a temperature sensor, temperatures of the pharmaceutical product; calculating, with at least one processor, changes between the detected temperatures over time; and generating, with at least one indicator and if a change of the calculated changes between the detected temperatures over time meets or falls below a predefined threshold, an indication that the pharmaceutical product is ready to be used.

In some embodiments, the predefined threshold is selected to correspond to when the active pharmaceutical ingredient (API) within the pharmaceutical product will be ready to be used.

In some embodiments, calculating changes between the detected temperatures over time comprises determining a difference between a first temperature and a second temperature, the first and second temperature detected a predefined amount of time apart.

In some embodiments, the at least one processor generates a ratio of the difference between the first and second temperature over the predefined amount of time apart and compares the ratio to the predefined threshold.

In some embodiments, a liquid crystal sensor configured to be attached to a pharmaceutical product that contains an active pharmaceutical ingredient (API) therein is provided, the liquid crystal sensor comprising: liquid crystals configured to change color to indicate increasing temperatures of the pharmaceutical product as the pharmaceutical product warms; at least one indicator configured to indicate when the API within the pharmaceutical product reaches a specified temperature or temperature range, wherein the specified temperature or temperature range is indicative of when the pharmaceutical product is ready to be used.

In some embodiments, a portion of the liquid crystals are adjacent to the at least one indicator on the liquid crystal sensor and are configured to change color when the pharmaceutical product is ready to be used.

In some embodiments, the liquid crystals comprise a visual meter that indicates the increasing temperatures of the pharmaceutical product as the pharmaceutical product warms.

In some embodiments, the at least one indicator corresponds with an end of the visual meter.

In some embodiments, the at least one indicator is configured to indicate an expected duration for the injection.

In some embodiments, any one or more of the features, characteristics, or elements discussed above with respect to any of the embodiments may be incorporated into any of the other embodiments mentioned above or described elsewhere herein.

### BRIEF DESCRIPTION OF THE FIGURES

FIG. 1 shows a pharmaceutical product with a detector disposed thereon for detecting temperatures of and indicating when a pharmaceutical product is ready to be used, according to some embodiments.

FIG. 2A shows a system diagram of a detector for detecting temperatures of and indicating when a pharmaceutical product is ready to be used, according to some embodiments.

FIGS. 2B-2C shows example detectors for indicating when a pharmaceutical product is ready to be used, according to some embodiments.

FIG. 2D shows an example graphical user interface (GUI) of a personal computing device for indicating when a pharmaceutical product is ready to be used, according to some embodiments.

FIG. 2E shows an example printed circuit board (PCB) of a detector for detecting temperatures of and indicating when a pharmaceutical product is ready to be used, according to some embodiments.

FIG. 2F shows a schematic of a detector for detecting temperatures of and indicating when a pharmaceutical product is ready to be used, according to some embodiments.

FIG. 3 shows a plot of time for the outside surface of a pharmaceutical product casing and the liquid housed within the casing to warm to a room temperature (e.g., 21.7° C.), according to some embodiments.

FIGS. 4A-4E show detectors comprising a fluid and one or more substrates for detecting temperatures of and indicating when a pharmaceutical product is ready to be used, according to some embodiments.

FIGS. 5A-5H show liquid crystal sensors for detecting temperatures of and indicating when a pharmaceutical product is ready to be used, according to some embodiments.

FIG. 6 shows a method for detecting temperatures of and indicating when a pharmaceutical product is ready to be used, according to some embodiments.

### DETAILED DESCRIPTION

Described herein are detectors configured to be attached to pharmaceutical products to detect temperatures of the pharmaceutical product and indicate if the pharmaceutical product is ready to be used based on a specified amount of time elapsing. The disclosed detectors may detect when a pharmaceutical product reaches a breach temperature and indicate if the pharmaceutical product is ready to be used based on a specified amount of time elapsing once the breach temperature is reached or exceeded. Breach temperatures may be defined as temperatures outside the standard refrigeration range (e.g., 2° C. to 8° C.). In some embodiments, a breach temperature may be between 10° C. and 15° C. (e.g., 12° C.). Based on the pharmaceutical product reaching one or more breach temperatures, the detector may start a timer configured to measure a passage of time and indicate that the pharmaceutical product is ready to be used after a specified amount of time has elapsed.

Traditionally, a user may refer to instructions for use (IFUs) that accompany a pharmaceutical product (e.g., on a product label and/or package insert) to determine how and when to use the pharmaceutical product. Additionally, a medical professional may inform the user instructions for use of a pharmaceutical product verbally in a medical visit and/or in a written medical note. Instructions may include a required wait time prior to using a pharmaceutical product. The wait time may be defined as the duration of time that a pharmaceutical product stored in a controlled cooling environment (e.g., refrigerator, freezer, etc.) is required to rest in an ambient environment (e.g., standard room temperature, around 20° C. to 22° C.) before use. The wait time may be required to allow the API of the pharmaceutical product to warm to an ambient temperature, thus decreasing the viscosity of the API to prepare the API for proper injection. The instructions may also include a maximum duration of time that the pharmaceutical product may not exceed while resting in an ambient environment. APIs may expire after a predefined duration of time outside of a controlled cooling environment (e.g., between 4 and 8 hours), thus making the API unsafe for use.

These instructions are often forgotten, misplaced, confused (e.g., with another pharmaceutical product), or ignored (e.g., if the user is in a hurry) during use of a pharmaceutical product. The user may attempt to use the pharmaceutical product directly after it is removed from the cooling environment, and in the instance the pharmaceutical product is one which requires injection, the inability to adhere to the wait time may cause a prolonged injection time, inability to inject the product, and/or pain at least at the injection site. Each of these issues may be due at least to the cool temperature and therefore high viscosity of the API. The injection time may be defined as the duration of time required to fully inject a pharmaceutical product into a user. In some instances, the user may forget to track the time after removing the pharmaceutical product from the controlled cooling environment and leave the product in the ambient environment for a duration of time that exceeds the maximum duration of time the product can be left outside of the cooling environment. In this instance, the pharmaceutical product can lose its efficacy and/or expire, thus making the product unsuitable and unsafe for use.

In some instances, despite the user adhering to the recommended wait time provided in the IFU and/or informed by a medical professional, the pharmaceutical product may require more or less time to reach the appropriate temperature for use. This variation in time to reach the appropriate use temperature may be due at least in part to variable room temperatures that the product is required to rest in before use. In certain instances, the pharmaceutical product may never reach an appropriate temperature for use. Currently, there is no way for the user to know that the pharmaceutical product is ready to use in advance of the instructed wait time or needs additional resting time prior to using. The detectors described herein detect temperatures of the pharmaceutical product and activate one or more timers based on at least one breach temperature being detected. The timers may be configured using various algorithms to indicate when the pharmaceutical product is ready to be used based on time elapsed and/or temperature reached. The provided detectors may more efficiently and effectively inform the user when a pharmaceutical product is ready to be used, alleviate any risk of user confusion, and improve user adherence to the wait time associated with the active pharmaceutical ingredient (API).

The detectors described herein may comprise a temperature sensor, at least one timer, and at least one indicator. In some embodiments, the temperature sensor may detect the temperature of the pharmaceutical product, and a first timer may start measuring a passage of time if the temperature sensor detects that the pharmaceutical product has reached or exceeded a breach temperature. In some embodiments, a second detector may be configured to measure a second passage of time if the temperature sensor detects that the pharmaceutical product has reached a second breach temperature. The at least one indicator may generate an indication that the pharmaceutical product is ready to be used when a specified amount of time measured by either the first or second timer has elapsed. In some embodiments, the detector may require user activation to begin detecting the temperature of the pharmaceutical product. In some embodiments, the indicator may include one or more illuminators configured to illuminate when the pharmaceutical product is not ready to be used and when the pharmaceutical product is ready to be used. In some embodiments, the sensor may be communicatively coupled with a personal computing device (e.g., desktop, tablet, mobile device, smart watch, etc.) to indicate to the user when the pharmaceutical product is/is not ready to be used.

In some embodiments, the detector may comprise a fluid that travels through one or more fluid pathways (e.g., timers) if the temperature of the fluid reaches or exceeds one or more breach temperatures. The fluid may travel through the one or more fluid pathways to an indicator for a specified amount of time to indicate that the pharmaceutical product is ready to be used. In some embodiments, the detector may require user activation for the fluid to travel to the temperature sensor.

In some embodiments, the detector may be embodied in a liquid crystal sensor comprising liquid crystals that may be configured to change colors based on an increasing temperature of the pharmaceutical product and indicate after a passage of time if the pharmaceutical product is ready to be used.

The detectors described herein may be associated with a variety of pharmaceutical products. For example, the pharmaceutical product may include drug delivery devices (e.g., autoinjectors, syringes, etc.), nasal sprays, nebulizers, eyedroppers, or containers (e.g., vials, bottles, cartridges, etc.) containing or configured to contain an active pharmaceutical ingredient (API) (e.g., pharmaceutical, medication, antibiotic, vaccine, drug, etc.). Injection devices may comprise syringes (e.g., hypodermic syringes, oral delivery syringes, pre-filled syringes, glass syringes, plastic syringes, etc.), on-body delivery systems (OBDS), patch pumps, auto-injector systems (e.g., pumps, patch pumps, and pens), manual injector systems, syringe pumps, etc.

In some embodiments, the pharmaceutical product may be provided with the detector attached to the product. In some embodiments, a user may be required to attach the detector to the pharmaceutical product before using the product. In some embodiments, the detector and pharmaceutical product may be single-use and disposable. In some embodiments, the detector may be reusable. In some embodiments, the pharmaceutical product may comprise an API contained in a container (e.g., vial, bottle, cartridge, etc.), and the user may remove the API from the container with an injection device when the detector associated with the container indicates that the pharmaceutical product is ready to be used.

In some embodiments, the active pharmaceutical ingredient (API) may require storage in a cool environment (e.g., refrigerator, freezer, etc.) up until the time of use. Example APIs may include eye/ear drops, reconstituted antibiotics, injections, and other large-molecule drugs in liquid form (e.g., Nipocalimab).

The list of APIs provided above is not intended to be exhaustive and may extend to any pharmaceutical, drug, medication, antibiotic, vaccine, etc. not explicitly stated herein. One of ordinary skill in the art would be able to reasonably apply the detectors disclosed herein to various pharmaceutical products, including various containers, injectors, etc. and APIs contained therein.

Referring now to the drawings, like parts are marked throughout the specification and drawings with the same reference numerals, respectively.

Pharmaceutical Products for Use with Detectors

The detectors disclosed herein may be configured to attach to a variety of pharmaceutical products containing an active pharmaceutical ingredient (API).

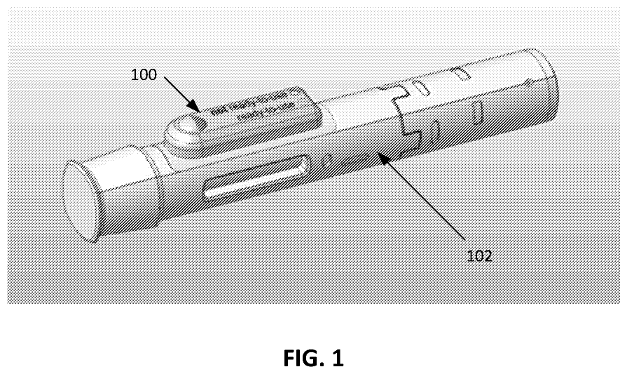

FIG. 1 shows an example pharmaceutical product with a detector 100 attached thereon. The pharmaceutical product 102 may comprise, for example, drug delivery devices, nasal sprayers, nebulizers, eyedroppers, containers, or another vessel for containing an API. As mentioned above, example drug delivery devices may include injection devices, such as syringes (e.g., hypodermic syringes, oral delivery syringes, pre-filled syringes, glass syringes, plastic syringes, etc.), on-body delivery systems (OBDS), patch pumps, auto-injector systems (e.g., pumps, patch pumps, and pens), manual injector systems, syringe pumps, etc. In some embodiments, example containers may include vials, bottles, cartridges, etc. The containing portion (e.g., drug delivery device, containers, etc.) of the pharmaceutical product described above may hereinafter be referred to as “containers.”

In some embodiments, detector 100 may be configured within the packaging of the pharmaceutical product 102. In some embodiments, detector 100 may be configured on an external portion of the packaging of the pharmaceutical product 102. In some embodiments, at least a portion of detector 100 may be configured within a reservoir of the pharmaceutical product 102, such that detector 100 may physically contact the API contained within the pharmaceutical product. In each embodiment wherein at least a portion of detector 100 (e.g., the temperature sensor) is not in contact with the API of the pharmaceutical product, one or more algorithms may be configured to extrapolate the temperature of the API based on the temperature of the container, packaging, etc.

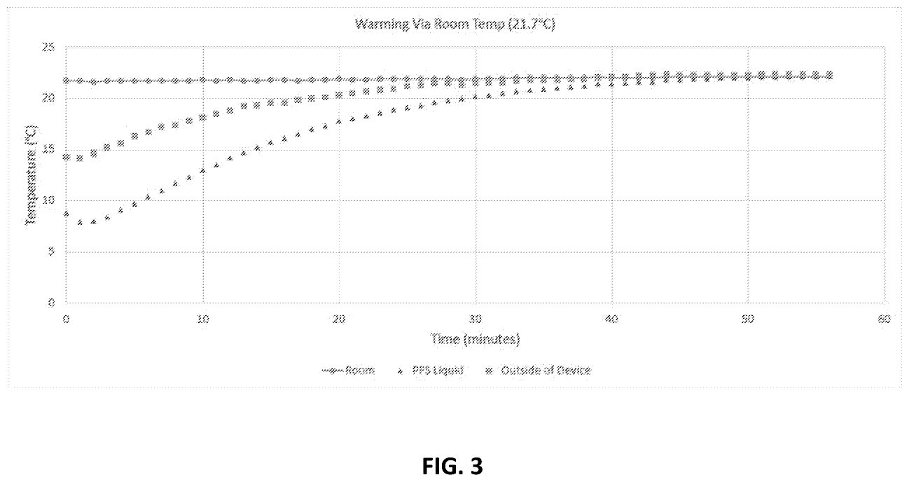

For example, detector 100 may be configured to detect temperatures of the outer surface of the pharmaceutical product 102 to determine the temperature of the API contained within the container, as will be described in greater detail at least with regards to FIG. 3. In some embodiments, detector 100 may be attached to the packaging of the pharmaceutical product or an outer surface of the pharmaceutical product and may be configured to detect the temperature of the environment (e.g., air) directly surrounding the pharmaceutical product to determine the temperature of the API. In some embodiments, detector 100 may be configured to attach to and detect temperatures of containers comprising one or more materials, such as glass and polymers (e.g., polyvinylchloride, polystyrene, polypropylene, polyethylene, polyester, nylon, polyvinylidene chloride, polycarbonate etc.). Many of these materials may be insulators; thus, via experimentation and/or algorithms, the temperature of the API within the container may be determined by considering properties of the heat transfer between the container, environment, and API, as will be described in greater detail below.

One or more of detector 100 and the container of pharmaceutical product 102 may comprise an adhesive material such that the detector 100 may be adhered to the pharmaceutical product 102, for example by a user and/or prior to providing the pharmaceutical product to the user. In some embodiments, the pharmaceutical product 102 and detector 100 may be manufactured and/or provided as a single unit, such that the detector 100 is not configured to be removable from the pharmaceutical product 102. In some embodiments, detector 100 may be provided on a removable sleeve, the sleeve configured to attach around at least a portion of the pharmaceutical product 102. In some embodiments, as mentioned above, the pharmaceutical product 102 may be single-use and disposable. Likewise, the detector 100 may be single-use and disposable. In some embodiments, each of the detector 100 and/or pharmaceutical product 102 may be reusable and/or semi-reusable. For example, a user may adhere a single reusable detector 100 to a different single-use pharmaceutical product 102 each day (or every few hours, days, months, etc.).

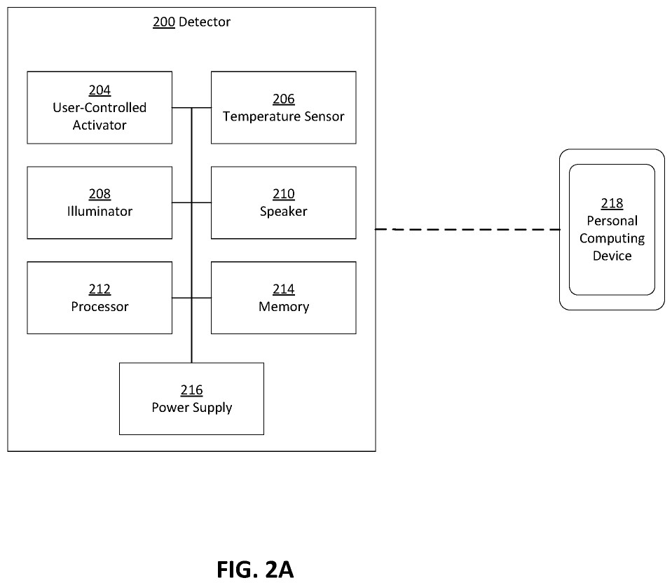

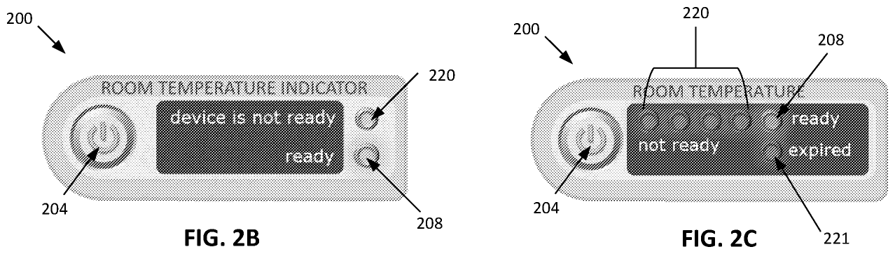

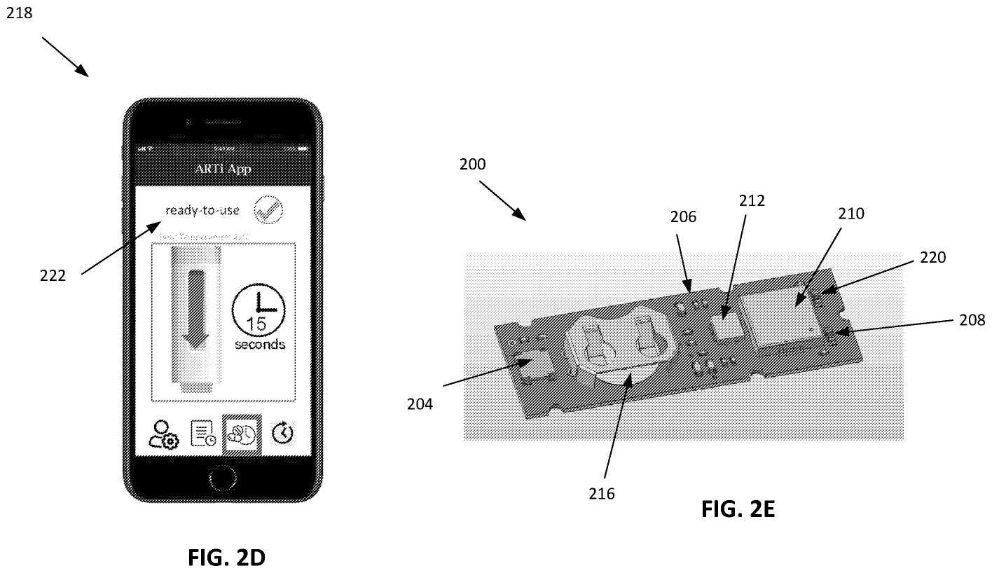

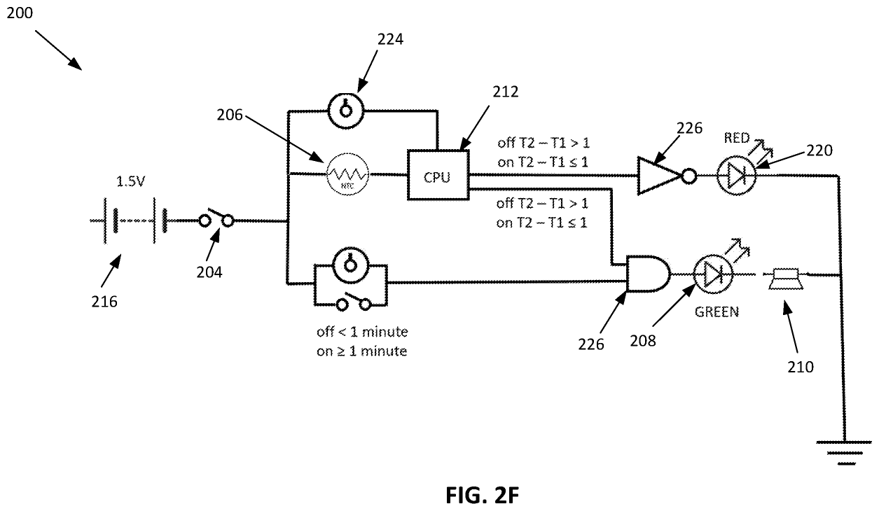

Detector 100 may be embodied in an electrical system, a fluid-based system, or a liquid crystal sensor system, each of which will be described in greater detail below with respect to FIGS. 2A-2F, FIGS. 4A-4E, and FIGS. 5A-5H.

### Electrical Detectors for Pharmaceutical Products

In some embodiments, detector 100 described above with respect to FIG. 1 may be embodied in an electrical detection system that includes one or more electrical components (e.g., one or more processors, temperature sensors, illuminators, etc.) communicatively coupled (e.g., via wired and/or wireless communication) to detect the temperature of the pharmaceutical product, measure the passage of time when one or more breach temperatures are reached or exceeded, and indicate when the pharmaceutical product is ready to be used based on the passage of time. FIGS. 2A-2F show features of an electrical detector configured to detect the temperature of and indicate if a pharmaceutical product is ready to be used based on a specified passage of time, according to some embodiments.

FIG. 2A shows a system diagram of a detector 200 configured to detect the temperature of a pharmaceutical product and indicate when the pharmaceutical product is ready to be used. Detector 200 may comprise any one or more features of detector 100 described above with respect to FIG. 1. Detector 200 may comprise a user-controlled activator 204, a temperature sensor 206, an illuminator (e.g., light indicator) 208, a speaker 210, a processor 212, a memory 214, and a power supply (e.g., battery) 216. Each of the illustrated components of detector 200 may be embodied by more than one component; for example, detector 200 may comprise multiple processors 212, temperature sensors 206, illuminators 208, etc. In some embodiments, each of the components of detector 200 may be communicatively coupled to one another, such that each component may transmit and receive signals to and/or from the other components of detector 200. In some embodiments, each of the components may be communicatively coupled to at least one or more processors 212 such that processor 212 is configured to receive signals from each of the components of detector 200 and, based on the received signals, transmit one or more signals to a component of detector 200 to perform an action.

In some embodiments, based on a received signal, processor 212 may be configured to activate and/or deactivate one or more components illustrated in detector 200 (e.g., temperature sensor 206, illuminator 208, and/or speaker 210). In some embodiments, processor 212 may comprise at least one timer configured to measure a passage of time (e.g., once a breach temperature is detected by the temperature sensor 206). The timer may, in some embodiments, measure time starting at zero and counting to a pre-defined time. In some embodiments, the timer may measure time starting at a pre-defined time and counting down to zero. In some embodiments, the timer is a separate component communicatively coupled at least with processor 212 and/or one or more components of detector 200 (e.g., temperature sensor 206). In some embodiments, processor 212 may comprise a plurality of timers configured to measure different passages of time to indicate when the pharmaceutical product is ready to be used after a specified amount of time has elapsed. In some embodiments, processor 212 may comprise one or more memories, such as memory 214 (e.g., memory 214 may be within processor 212).

As shown in FIG. 2A, detector 200 may comprise a user-controlled activator 204. User-controlled activator 204 may comprise one or more touch-sensitive buttons, switches, capacitive touch sensors, pressure sensors, etc. configured to receive a user input indicative of a request to activate (e.g., turn on) detector 200. For example, FIG. 2B illustrates detector 200 comprising user-controlled activator 204, wherein the activator may be a touch-sensitive button. In some embodiments, user-controlled activator 204 may be configured to transmit a signal (e.g., directly and/or indirectly via processor 212) to one or more components of detector 200 (e.g., temperature sensor 206, illuminator 208, and/or speaker 210) upon receipt of a user input. For example, user-controlled activator 204 may be configured to receive the user input and transmit a signal to temperature sensor 206 to cause temperature sensor 206 to activate and begin detecting the temperature of the pharmaceutical product. In some embodiments, the user-controlled activator 204 may instead or additionally transmit a signal to processor 212 such that the processor may transmit a signal to temperature sensor 206 to activate the sensor. A user may engage with (e.g., tap, push, etc.) the user-controlled activator 204 for a specified amount of time (e.g., 1, 2, 3 seconds) to cause detector 200 to activate. In some embodiments, in response to a user activating detector 200 via user-controlled activator 204, a light indicator (e.g., illuminator 220 in FIG. 2B) may be configured to illuminate (e.g., based on a signal received from user-controlled activator 204 and/or processor 212). The illuminator may communicate, for example, that detector 200 is active (e.g., at least detecting the temperature of pharmaceutical product), but not yet ready to be used.

In some embodiments, detector 200 may comprise one or more temperature sensors 206. Temperature sensor 206 may be configured to continuously detect the temperature of the pharmaceutical product (e.g., pharmaceutical product 102 described above with respect to FIG. 1). In some embodiments, temperature sensor 206 may be disposed within detector 200 such that at least a portion of temperature sensor 206 is adjacent to and/or contacts the outer surface of the pharmaceutical product. As described above, in some embodiments, temperature sensor 206 may be disposed on and/or configured to measure a temperature of the packaging of the pharmaceutical product. In some embodiments, temperature sensor 206 may be disposed within the pharmaceutical product to detect temperatures of the API directly. In some embodiments, temperature sensor 206 may be configured to detect temperatures of the environment (e.g., air) directly surrounding the pharmaceutical product. Temperature sensor 206 may comprise one or more thermocouples, resistance temperature detectors (RTDs), thermistors (e.g., negative temperature coefficient (NTC) thermistor), and/or semiconductor-based integrated circuits (IC). For example, temperature sensor 206 may comprise an NTC thermistor, such that when temperature increases, the resistance of the thermistor decreases. In some embodiments, the thermistor may comprise a polymer or ceramic material. Temperature sensor 206 may be communicatively coupled to one or more components of detector 200. For example, temperature sensor 206 may be configured to receive an input (e.g., signal) from user-controlled activator 204 (e.g., directly and/or indirectly via processor 212). For example, as described above, user-controlled activator 204 may transmit a signal to temperature sensor 206, and temperature sensor 206 may activate based on the received signal.

In some embodiments, temperature sensor 206 may be configured to transmit and receive signals to/from processor 212. For example, temperature sensor 206 may continuously detect the temperature of the pharmaceutical product as the pharmaceutical product warms to an appropriate use temperature (e.g., towards an ambient temperature). For each detected temperature, temperature sensor 206 may transmit a signal indicative of the detected temperature to processor 212. The processor 212 may determine when the pharmaceutical product is ready to be used based on the detected temperature and/or a passage of time, as will be described in greater detail below.

In some embodiments, upon detection of a breach temperature of the pharmaceutical product, temperature sensor 206 may be configured to cause (e.g., directly and/or indirectly via processor 212) activation of one or more timers. The breach temperature of the pharmaceutical product may be, as defined above, a temperature outside of the standard refrigeration range (e.g., 2° C. to 8° C.). In some embodiments, the breach temperature may be between 10° C. and 15° C. (e.g., 12° C.), such that minute fluctuations in temperature above the standard refrigeration range for short periods of time may not be classified as a breach temperature. In some embodiments, the initial temperature detected by temperature sensor 206 may be the breach temperature. In some embodiments, a value for the breach temperature may be stored in a temperature library of memory 214, such that a processor 212 may retrieve temperature data from memory 214 to classify the signal as corresponding to the detected breach temperature.

In some embodiments, the timer may measure the passage of time after detection of the breach temperature for a specified amount of time. The specified amount of time may be based on one or more factors, such as the material of the container of the pharmaceutical product, the ambient room temperature detected, and/or the API contained in the container. For example, memory 214 may be configured to store a library of specific times associated with different pharmaceutical product containers, APIs, and room temperatures. The system may be configured to determine the room temperature to select a specific amount of time, for example, using at least the detected temperature of the container, as will be described in greater detail below. In some embodiments, as mentioned above, the timer of processor 214 may be configured to count from zero up to the specified time. In some embodiments, the timer may be configured to count down from the specified time towards zero.

In some embodiments, temperature sensor 206 may continue to detect the temperature of the pharmaceutical product following detection of the breach temperature. In some embodiments, temperature sensor 206 may detect a second breach temperature greater than the first breach temperature. Based on the second breach temperature, temperature sensor 206 (e.g., directly or indirectly via processor 212) may be configured to cause the timer to measure a second passage of time. In some embodiments, the second passage of time may be less than the first passage of time. In some embodiments, detector 200 may comprise more than one timer (e.g., 2, 3, 4, 5 or more timers) configured to measure each passage of time of interest (e.g., optionally embodied in processor 212). In some embodiments, rather than measuring a second passage of time, based on the second detected breach temperature, processor 212 may be configured to modify the specified amount of time that the timer is counting. For example, processor 212 may reduce the time that the timer is counting towards in the instance the timer is counting from zero. In some embodiments, processor 212 may reduce the current time of the timer in the instance the timer started counting time from a pre-defined value associated with the first breach temperature and/or pharmaceutical product based on the second detected breach temperature.

For example, detector 200 may be configured to indicate, if the detected temperatures are less than a breach temperature (e.g., 15° C. in this example), that the pharmaceutical product is not ready to be used. Upon temperature sensor 206 detecting a temperature greater than or equal to 15° C., processor 212 may be configured to start measuring a first passage of time. For example, the timer may measure a 30-minute passage of time, such that the pharmaceutical product will be ready to be used after 30 minutes has elapsed. In some embodiments, the temperature sensor 206 may continue to detect temperatures of the pharmaceutical product after the first timer has started measuring the passage of time. Upon temperature sensor 206 detecting a second breach temperature (e.g., 18° C. in this example), processor 212 may be configured to start measuring a second passage of time. For example, based on the second breach temperature, the timer may be configured to measure a 5-minute passage of time. Detector 200 may be configured such that the user is notified that the pharmaceutical product is ready to be used when either the first or second passage of time has elapsed (e.g., whichever occurs first).

In some embodiments, detector 200 may comprise a third timer configured to measure the passage of time regardless of detected breach temperatures. For example, the third timer may be configured to measure the passage of time once detector 200 is activated, and after a pre-defined amount of time has elapsed, may indicate that the pharmaceutical product is ready to be used.

In some embodiments, in addition to or instead of measuring the passage of time after a breach temperature has been reached and/or exceeded, processor 212 may be configured to determine a relationship between the passage of time and the detected temperatures to determine if a pharmaceutical product is ready to be used. For example, processor 212 may determine when the pharmaceutical product is ready to be used by continuously tracking the change in temperature of the pharmaceutical product over time. Processor 212 may analyze the ratio of temperature change over time, and if processor 212 detects a low, stable ratio (e.g., less than 1), processor 212 may cause one or more indicators (described in greater detail below) to indicate that the pharmaceutical product is ready to be used. For example, processor 212 may continuously compare two temperatures detected a predefined duration of time apart (e.g., 1 minute) to determine the ratio (e.g., 2^{nd} Temperature−1^{st} Temperature/1 minute). In some embodiments, processor 212 may compare two temperatures detected at a smaller duration of time apart (e.g., 15, 30, 45 seconds, etc.) or a greater duration of time apart (e.g., 2, 3, 4 minutes, etc.). Based on the relationship between temperature and time, processor 212 may be able to determine that the pharmaceutical product has equilibrated to an appropriate use temperature (e.g., ambient temperature) and is ready to be used.

FIG. 2F illustrates an example schematic with one or more logic gates for determining when the pharmaceutical product is ready to be used in accordance at least with the above algorithm. For example, upon a user engaging with the user-controlled activator (e.g., switch) 204, a timer 224 may be configured to begin measuring a passage of time, and a temperature sensor 206 (e.g., negative temperature coefficient (NTC) thermistor) may simultaneously begin detecting temperatures of the pharmaceutical product. The timer 224 (e.g., optionally embodied in processor 212) and temperature sensor 206 may transmit signals indicative of measured time and detected temperatures, respectively. Based on the received signals, the processor (e.g., computer processing unit (CPU)) 212 may be configured to analyze the change in temperature. As shown in FIG. 2F, based on the analysis, processor 212 may send a signal to one or more logic gates 226 communicatively coupled to one or more indicators. For example, when the temperature change is greater than 1, processor 212 may transmit an “off” (e.g., binary 0) signal to a “NOT” logic gate 226 (e.g., configured to do the opposite of the received signal) communicatively coupled to an illuminator (e.g., LED light indicator) 220. As will be described in greater detail below, illuminator 220 may be configured to indicate to the user when the pharmaceutical product is not ready to be used. Processor 212 may also (e.g., at substantially the same time) transmit the “off” signal to an “AND” logic gate. The “AND” logic gate may receive an input from an additional component of detector 200 configured to determine when detector 200 is stabilized. For example, upon activation of detector 200 with user-controlled activator 204, a second timer may be configured to begin measuring a pre-defined passage of time (e.g., at least 1 minute). Once the passage of time has elapsed, a switch may be actuated, such that a signal may be transmitted to the “AND” logic gate. Upon two matching signals (e.g., binary 1s) from the components, an illuminator (e.g., LED light indicator) 208 communicatively coupled to the “AND” logic gate may be activated. In some embodiments, when the temperature change is less than 1, processor 212 may transmit an “on” (e.g., binary 1) signal to the logic gates 226. The “NOT” logic gate may transmit the signal to illuminator 220 to deactivate the illuminator. On the other hand, the “AND” logic gate may, in receipt of two matching signals (e.g., binary 1s), transmit a signal to one or more indicators (e.g., illuminator 208, speaker 210) to indicate that the pharmaceutical product is ready to be used.

In some embodiments, processor 212 may use the initial temperature of the environment outside of the controlled, cooling environment (e.g., once the pharmaceutical product is removed from a refrigerator) to determine when the pharmaceutical product is ready to be used. For example, temperature sensor 206 may detect temperatures of the pharmaceutical product during an initial stabilization period occurring upon activation of detector 200 (e.g., via user-controlled activator 204). The initial stabilization period may be a predefined duration of time (e.g., 1 minute, 90 seconds, 2 minutes, etc.). For example, temperature sensor 206 may detect a first temperature at the activation time (time₁=0), and a second temperature at a predefined time after the activation time, wherein the predefined time is based on a computed time constant of detector 200 (e.g., time₂=0.7*time constant). The time constant of detector 200 may be determined experimentally by measuring the change in temperature of the pharmaceutical product detected by detector 200 and noting the point at which, for example, 63% settling has occurred. Using the first and second detected temperatures, processor 212 of detector 200 may estimate the ambient (e.g., room) temperature. For example, the relationship between the estimated ambient temperature and the detected first and second temperature may be embodied in the following formula:

$$\begin{matrix}Estimated ambient temperature & \\ {T}_{ambient} = {T}_{1} + 2 ( {T}_{2} - {T}_{1} ) . & Equation 1\end{matrix}$$

In some embodiments, the relationship between the first and second temperature to determine an ambient room temperature may be different from that provided above in equation 1. In some embodiments, one or more of the estimated ambient temperature, first temperature, and second temperature may be stored, for example, in a memory (e.g., a temporary memory of processor 212, memory 214, etc.). Based on the estimated ambient temperature, processor 212 may determine a target temperature (e.g., the temperature at which the pharmaceutical product will be ready to be used) and/or a maximum wait time (e.g., the maximum time required to wait before using the pharmaceutical product). In some embodiments, the target temperature may be a fraction (e.g., 90%, 95%, 98%, or another specified value) of the estimated ambient temperature. In some embodiments, the maximum wait time may additionally or instead be based on the type of API within the container, the material of the container, the volume of API, etc. For example, as described above, processor 212 may access a memory 214 storing target temperatures associate with a determined ambient temperature and maximum wait times associated with one or more of a determined wait time and one or more factors provided above.

In some embodiments, processor 212 may begin measuring the passage of time (e.g., bound by the maximum wait time) upon a determination of the estimated ambient temperature. In some embodiments, the maximum wait time may be independent of the estimated room temperature, such that a timer of processor 212 begins measuring the passage of time (bound by a predetermined maximum wait time) upon activation of detector 200. Upon a determination of the target temperature, temperature sensor 206 may be configured to periodically detect the temperature of the pharmaceutical product over time (e.g., detecting at least every 5, 10, 20, 30, or 60 seconds). Processor 212 may be configured to receive signals from temperature sensor 206 indicative of the detected temperatures and may compare the detected temperatures to the target temperature. In some embodiments, processor 212 may be configured to activate one or more indicators upon a detection of a temperature equal to or greater than the target temperature, or upon the maximum wait time being reached, whichever occurs first.

In some embodiments, processor 212 may be configured to measure the passage of time upon activation of detector 200 (e.g., via user-controlled activator 204) until detection of a breach temperature (e.g., 12° C., 15° C., or another predefined temperature). Based on the amount of time elapsed to reach a breach temperature, processor 212 may determine the ambient (e.g., room) temperature and/or the amount of time required for the pharmaceutical product to reach an appropriate use temperature. For example, as described above, the temperature sensor 206 may periodically detect temperatures of the pharmaceutical product and compare the detected temperatures to the determined appropriate use temperature to identify if the pharmaceutical product is ready to be used. In some embodiments, temperature sensor 206 may cause one or more timers (e.g., embodied in processor 212) to activate and measure a passage of time based on the determined amount of time required for the pharmaceutical product to reach an appropriate use temperature.

In some embodiments, processor 212 may be configured to measure a specified amount of time upon activation of detector 200, regardless of the temperature of the environment outside the controlled cooling environment.

In some embodiments, processor 212 may apply one or more methods (e.g., algorithms) described above to determine when the pharmaceutical product is ready to be used. For example, processor 212 may use a combination of algorithms and provide an indication that the pharmaceutical product is ready to be used when it is determined that a scenario described above has occurred (e.g., whichever occurs first may cause an indication).

In some embodiments, detector 200 may comprise one or more indicators configured to indicate that the pharmaceutical product is ready to be used. As mentioned above, detector 200 may additionally include one or more indicators configured to indicate when the pharmaceutical product is not ready to be used (e.g., illuminator 220 illustrated in FIGS. 2B-2C). In some embodiments, the one or more indicators of detector 200 may comprise an illuminator 208 and/or a speaker 210. In some embodiments, illuminator 208 may comprise one or more light emitting diodes (LEDs). In some embodiments, illuminator 208 may be configured to receive a signal transmitted from processor 212, the signal indicative that the pharmaceutical product is ready to be used. For example, processor 212 may be configured to transmit a signal to illuminator 208 when a specified amount of time, mentioned above, has elapsed. In some embodiments, processor 212 may be configured to transmit a signal to illuminator 208 upon a determination that an appropriate use temperature has been reached (e.g., prior to the specified time elapsing). In some embodiments, processor 212 may cause the illuminator, which is configured to indicate to the user that the pharmaceutical product is not ready to be used (e.g., illuminator 220 in FIGS. 2B-2C), to deactivate when the specified time has elapsed and/or when the appropriate use temperature is reached. In some embodiments, illuminator 220 may be configured to deactivate at substantially the same time that illuminator 208 is activated.

In some embodiments, illuminator 208 may be configured to illuminate for a pre-defined duration of time. For example, illuminator 208 may illuminate for any duration of time (e.g., seconds, minutes, or hours) up to a specified amount of time (e.g., 4, 5, 6, 7, or 8 hours). In some embodiments, a timer of processor 212 may measure the amount of time that the pharmaceutical product has been ready to be used and may indicate after a predefined duration of time being ready (e.g., 4 hours), that the pharmaceutical product should not be used. For example, after the predefined duration of time, illuminator 208 may be deactivated (e.g., neither illuminator 208 nor illuminator 220 are activated). In some embodiments, detector 200 may comprise an additional indicator (e.g., illuminator 221 in FIG. 2C) configured to illuminate after a predefined duration of time to indicate that the pharmaceutical product is expired. For example, various APIs may not be safe for use after resting outside of a controlled cooling environment for a predefined duration of time (e.g., 2, 4, 6, 8 hours). Illuminator 221 may be configured to illuminate once a predefined duration of time has elapsed, the duration of time corresponding with the API of the pharmaceutical product. By deactivating illuminator 208 and/or activating illuminator 221 after a predefined duration of time, detector 200 may indicate the window of time in which it is safe to use the pharmaceutical product.

In some embodiments, illuminator 208 may be communicatively coupled (e.g., directly or indirectly via processor 212) to user-controlled activator 204, such that a user may engage with user-controlled activator 204 to deactivate illuminator 208. In some embodiments, a user may engage with user-controlled activator 204 to pause and/or power off detector 200. In some embodiments, detector 200 may comprise one or more sensors (not illustrated) configured to detect when the pharmaceutical product has been and/or is currently being used, and the illuminator 208 may be configured to deactivate upon a determination that the pharmaceutical product has been used.

In some embodiments, illuminator 208 (and/or illuminator 220 illustrated in FIGS. 2B-2C) may be configured to continuously illuminate and/or flash. For example, the illuminators may flash in accordance with a pre-defined pattern corresponding with the passage of time as the pharmaceutical product nears a ready-to-use state. For example, illuminator 220 may initially flash at a slow rate, and as the pharmaceutical product reaches a time and/or temperature appropriate for use, the rate at which the illuminator flashes may increase. In some embodiments, illuminator 220 may initially flash at a fast rate, and may reduce in rate as the pharmaceutical product approaches a ready to use state (e.g., based on measured temperature and/or time elapsed).

In some embodiments, detector 200 may comprise an array of illuminators (e.g., illustrated in FIG. 2C) configured to indicate the state of the pharmaceutical product as it reaches an appropriate temperature and/or time for use. For example, detector 200 may comprise at least 2, 3, 4, 5, 6, 7, or 8 illuminators configured to indicate a state of the pharmaceutical product (e.g., not ready, ready, and/or expired). In some embodiments, an array of illuminators 220 may be configured to progressively illuminate as time elapses and/or the temperature of the pharmaceutical product nears an appropriate use temperature. For example, a first illuminator may activate when the detector 200 is activated, a second illuminator may activate a specified amount of time following activation of the detector and the first illuminator, a third illuminator may activate a specified amount of time following activation of the second illuminator, etc. until the illuminator 208 corresponding with when the device is ready to be used is activated. In some embodiments, the plurality of illuminators 220 may be configured to progressively illuminate as breach temperatures are detected. For example, if a first breach temperature is detected, a first illuminator may activate; if a second breach temperature is detected, a second illuminator may activate; if a third breach temperature is detected a third illuminator may activate, etc. until an appropriate use temperature and/or specified passage of time is reached. In some embodiments, the illuminators may comprise one or more colors (e.g., red, blue, green, white, yellow, etc.). For example, illuminator 208 may illuminate a first color (e.g., green), and illuminator 220 may illuminate a second color (e.g., red) different from the first color.

As shown in FIGS. 2B-2C, each of the illuminators 208, 220, and 221 may correspond with a text label and/or icon on detector 200 to indicate if the pharmaceutical product is/is not ready to be used. For example, detector 200 may comprise a label (e.g., “not ready to use,” “not ready,” “active,” “on,” etc.) adjacent to illuminator 220 configured to indicate that detector 200 is active but the pharmaceutical product is not yet ready to be used. Likewise, detector 200 may additionally comprise a label (e.g., “ready to use,” “ready,” etc.) adjacent to illuminator 208 configured to indicate that the pharmaceutical product is ready to be used. Detector 200 may additionally comprise a label (e.g., “expired,” “lapsed,” etc.) adjacent to illuminator 221 configured to indicate that the pharmaceutical product has expired, e.g., due to the amount of time outside of the controlled cooling environment reaching or exceeding the maximum amount of time allotted.

In some embodiments, in addition to or in place of activating illuminator 208, processor 212 may transmit a signal to speaker 210 configured to indicate that the pharmaceutical product is ready to be used. In some embodiments, speaker 210 may comprise a multi-tone and/or piezoelectric sounder (e.g., buzzer). Speaker 210 may generate an audible sound (e.g., tone, alarm, noise, etc.) in response to receiving a signal from processor 212. In some embodiments, speaker 210 may produce a continuous or discontinuous (e.g., beeping pattern) sound. In some embodiments, speaker 210 may be configured to produce a sound for a predefined duration of time. For example, speaker 210 may produce a sound for less than or equal to 5, 10, 15, 20, 25, or 30 seconds. In some embodiments, speaker 210 may produce a noise for greater than or equal to 5, 10, 15, 20, 25, or 30 seconds. In some embodiments, speaker 210 may be communicatively coupled (e.g., directly or indirectly via processor 212) to user-controlled activator 204, such that a user may engage with user-controlled activator 204 to deactivate speaker 210. In some embodiments, detector 200 may comprise one or more sensors (not illustrated) configured to determine when the pharmaceutical product has been and/or is currently being used and based on the determination that the pharmaceutical product is used, the speaker 210 may be configured to deactivate. In some embodiments, detector 200 may comprise one or more volume controllers (not illustrated) such that a user may control the volume at which speaker 210 produces sound.

As shown in FIG. 2A, detector 200 may be communicatively coupled via wireless communication (e.g., WiFi, Bluetooth, Zigbee, etc.) to a personal computing device 218 for indicating when the pharmaceutical product is ready to be used. Personal computing device 218 may comprise a mobile device, tablet, desktop, smart watch, etc. In some embodiments, detector 200 may comprise one or more processors configured to transmit and/or receive signals from personal computing device 218 (i.e., distinct from processor 212 which may be configured to transmit and/or receive signals from components within detector 200). FIG. 2D illustrates an example mobile computing device 218 with a graphical user interface (GUI) 222. As shown, GUI 222 of mobile computing device 218 may be configured to indicate when the pharmaceutical product is ready to be used. In some embodiments, GUI 222 may be configured to indicate when the pharmaceutical product is not ready to be used. GUI 222 may provide the detected temperature (e.g., temperature of the container) when the pharmaceutical product is/is not ready to be used. In some embodiments, mobile computing device 218 may be configured to generate a notification when the pharmaceutical product is ready to be used (e.g., based on temperature of the pharmaceutical product and/or elapsed time). For example, mobile computing device 218 may generate a notification (e.g., a sound, vibration, text notification, etc.) when the pharmaceutical product is ready to be used, despite the mobile computing device not currently displaying GUI 222. In some embodiments, GUI 222 may additionally provide information to a user related to the API (e.g., type of API and/or dose, expiration date, etc.).

In some embodiments, GUI 222 may display the expected injection time. The injection time may be defined as the expected duration of time for injecting an API, for example, in the instance the pharmaceutical product comprises an autoinjector. The injection time may, for example, dynamically update (e.g., decrease) as the autoinjector injects the API into the user. In some embodiments, processor 212 may comprise one or more sensors configured to detect when an autoinjector has started and/or finished injecting, and this data may be transmitted to mobile computing device 218 for display on GUI 222. In some embodiments, an autoinjector may experience a plunger delay (e.g., time difference between detector 200 detecting that injection is complete and the end of the stopper travel in the autoinjector) based on the detected temperature that may be considered when determining when the injection has finished. In some embodiments, the injection data of a given patient may be tracked through GUI 222 and transmitted, for example, to an electronic health record (EHR) associated with the patient and accessible to a medical professional to allow the medical professional to view the patient's compliance with their dosing regime.

In some embodiments, detector 200 may additionally comprise a display (not illustrated) communicatively coupled to one or more components of detector 200 (e.g., processor 212) and configured to provide at least a portion of the information described above in relation to GUI 222 of mobile computing device 218. For example, detector 200 may comprise a liquid crystal display (LCD) that may display an expected injection time, detected temperatures of the pharmaceutical product, wait times, etc. In some embodiments, detector 200 may comprise one or more illuminators (e.g., illuminator 208, 220, 221, etc.) in combination with an LCD display.

FIG. 2E shows an example printed circuit board (PCB) implementation of detector 200. In some embodiments, user-controlled activator 204 may comprise a PCB switch. In some embodiments, processor 212 may comprise a micro-controller. In some embodiments, temperature sensor 206 may comprise a thermistor. The PCB of detector 200 may comprise a plurality of indicators. For example, detector 200 may comprise a speaker 210 (e.g., piezeoelectric sounder), an illuminator 208 (e.g., LED) configured to indicate when the pharmaceutical product is ready to be used, and an illuminator 220 (e.g., LED) configured to indicate when the pharmaceutical product is not ready to be used. In some embodiments, the power supply 216 of detector 200 may comprise a battery (e.g., single-use or rechargeable battery). In some embodiments, the battery may be configured to be removable from a retainer of the PCB, such that the battery may be removed after use of detector 200 (e.g., in the instance detector 200 and/or the pharmaceutical product are single-use). The PCB of detector 200 may be stored within a housing, the housing configured to be attached to the pharmaceutical product. In some embodiments, the PCB of detector 200 is embodied within the housing of the pharmaceutical product. An example housing is illustrated in FIG. 1 of detector 100 and pharmaceutical product 102.

An example use of electronic detector 200 is provided herein. In some embodiments, a user may remove the pharmaceutical product with detector 200 attached thereon from a controlled, cooling environment (e.g., refrigerator) and from the packaging of the pharmaceutical product and activate detector 200 via user-controlled activator 204, which may cause temperature sensor 206 to begin automatically detecting the temperature of the pharmaceutical product. Upon activation of the device, illuminator 220 may illuminate to indicate to the user that detector 200 is active, however, is not yet at an appropriate use temperature and therefore is not ready to be used. Additionally, upon detection of a first temperature (e.g., breach temperature) outside of the standard refrigeration range (which may occur at substantially the same time as illuminator 220 being activated), the timer may begin passively measuring the passage of time. Once a specified amount of time has elapsed, processor 212 (which may comprise the timer measuring the passage of time) may cause illuminator 208 to illuminate and illuminator 220 to deactivate. By activating illuminator 208, detector 200 may indicate to the user that the pharmaceutical product is within an appropriate temperature range for use. In some embodiments, the appropriate temperature range for use may correspond with the standard ambient (e.g., room) temperature range of 20° C. to 22° C. In some embodiments, the appropriate temperature range for use may range between 15° C. and 30° C., dependent at least on the API of the pharmaceutical product. In some embodiments, the user may additionally be notified that the pharmaceutical product is ready to be used from a sound indication produced by speaker 210 and/or via a notification on GUI 222 of mobile computing device 218.

### Example Relationship Between API and Container Temperature

FIG. 3 illustrates an example plot of temperature against time, wherein the temperature of a container, a temperature of the liquid (e.g., API) contained within the container, and the ambient room temperature (e.g., 21.7° C. in this example) are measured over time. The plot of FIG. 3 may demonstrate the relationship between the temperature of each of the three entities (e.g., liquid, container, and room). In some embodiments, the relationship between at least the temperature of the liquid and the container may be applied within one or more algorithms configured in detector 200, such that when detector 200 detects the temperature of the container, as described above, the temperature of the API within the container may be estimated (e.g., extrapolated) based on the plotted and/or tabulated relationship. As shown, the temperature of the container and liquid may increase at substantially the same rate over time, however the temperature of the liquid at may initially be cooler from storage in the controlled cooling environment. Thus, the container may reach room temperature sooner than the liquid, dependent on one or more factors such as the type of liquid, material of the container, volume of the liquid, presence of an air gap, etc. For example, based on the thermal capacity of the container (e.g., dependent at least on the mass and specific heat of the container), the rate at which heat is transferred from the ambient environment, through the container, and to the API may vary. As shown, the temperature differential between the temperature of the container and the temperature of the liquid may reduce over time. Thus, in some embodiments, it may not be required for the temperature of the container to reach the ambient room temperature for the pharmaceutical product to be ready to be used. This relationship may be determined and tabulated for a plurality of container materials, liquid types, and liquid volumes, for example, for use in algorithms that determine when the pharmaceutical product is ready to be used.

### Fluid-Based Detectors for Pharmaceutical Products

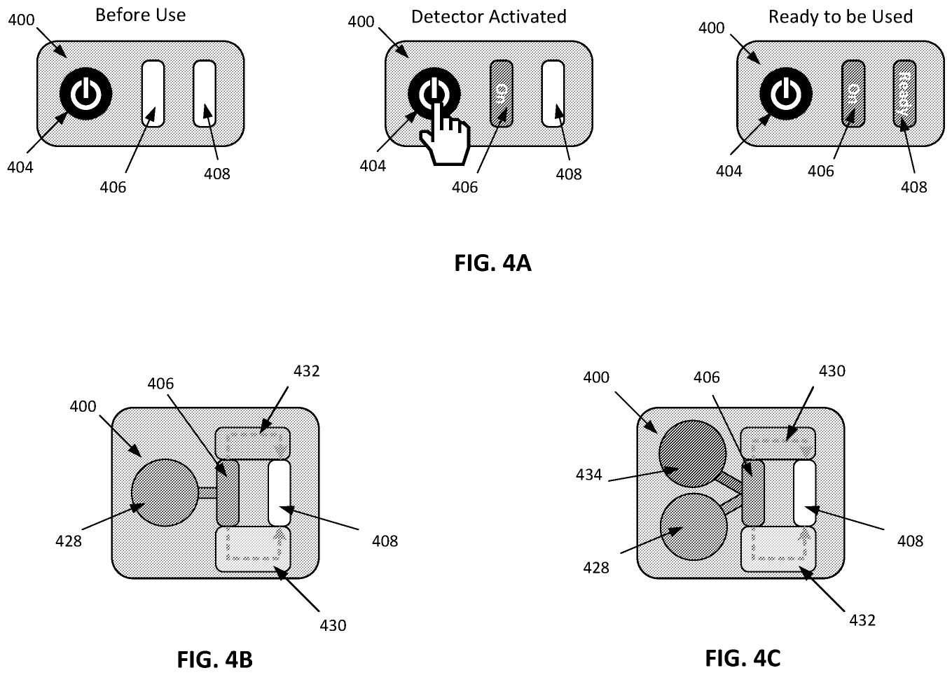

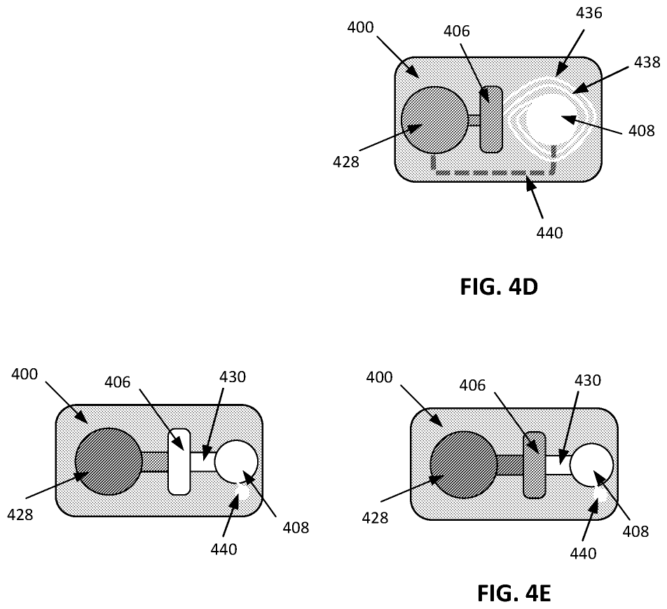

In some embodiments, detector 100 described above with respect to FIG. 1 may be embodied in a fluid-based system that includes one or more fluids (e.g., liquids, gases) that may be selected to detect a change in temperature of a pharmaceutical product and travel for a specified duration of time prior to indicating if the pharmaceutical product is ready to be used. FIGS. 4A-4E show various fluid-based detectors configured to detect the temperature of and indicate if a pharmaceutical product is ready to be used when a specified amount of time has elapsed, according to some embodiments.

FIG. 4A shows a front-end of a fluid-based detector 400 in different stages of use, the detector 400 configured to detect the temperature of and indicate if a pharmaceutical product is ready to be used after a specified amount of time has elapsed. Detector 400 may comprise any one or more features of detectors 100, 200 described above with respect to FIG. 1 and FIGS. 2A-2F. For example, detector 400 may be removably attached to a pharmaceutical product (e.g., with an adhesive). In some embodiments, detector 400 may be disposable and single-use due to one or more irreversible features. Detector 400 may comprise a user-controlled activator 404, a temperature sensor 406, and an indicator 408. In some embodiments, user-controlled activator 404 may comprise a button (e.g., blister) configured to activate movement of a fluid contained in a reservoir (e.g., sealed pod) associated with the button. The fluid may comprise a miscible fluid (e.g., dyed liquid, gel, etc.). In some embodiments, the temperature of the fluid of detector 400 may be related to (e.g., via one or more empirical relationships) the temperature of the active pharmaceutical ingredient (API) contained within the pharmaceutical product, such that the detected temperature of the fluid may be used to determine when the pharmaceutical product is ready to be used.

In some embodiments, temperature sensor 406 and indicator 408 may each comprise a window and a reservoir (e.g., chamber) such that a user may view a fluid (e.g., liquid) in the reservoir through the window to indicate the status of the detector 400. The windows of indicator 408 and temperature sensor 406 may comprise a circular, oval, rectangular, or other shape. In some embodiments, the windows of indicator 408 and temperature sensor 406 may comprise substantially the same shape; in some embodiments the window of temperature sensor 406 may be one shape (e.g., rectangle) and the window of indicator 408 may be a different shape (e.g., oval).

As shown, before use, the temperature sensor window and indicator window may be vacant (e.g., empty) reservoirs. Upon activation of detector 400, a fluid may travel from the reservoir associated with user-controlled activator 404 to the temperature sensor 406 to indicate that the detector is active. For example, user-controlled activator 404 may be configured to induce pressure in a reservoir containing the fluid of detector 400, such that when user-controlled activator 404 is pressed or pushed down (e.g., for 1, 2, 3, or more seconds), the fluid contained in the reservoir may be forced out of the reservoir and toward temperature sensor 406 (e.g., via one or more capillaries, as will be described in greater detail with respect to FIGS. 4B-4E). In some embodiments, the fluid contained in the reservoir associated with user-controlled activator 404 may be configured to begin traveling toward temperature sensor 406 upon a detected change in temperature that may affect the state of the fluid (e.g., at the melting point of the fluid, where the fluid changes from a solid to a liquid). After a specified amount of time, the fluid visible in temperature sensor 406 may travel to (e.g., via one or more capillaries, substrates, gels, etc.) and be visible in indicator 408 to indicate that the pharmaceutical product is ready to be used.

In some embodiments, the temperature sensor 406 may comprise a label configured to indicate that detector 400 is active, however the pharmaceutical product is not ready to be used (e.g., “on,” “active,” “not ready to be used,” “not ready,” etc.). Likewise, the indicator 408 may comprise a label configured to indicate that the pharmaceutical product is ready to be used (e.g., “ready,” “ready to be used,” “ready to use,” etc.). Each of the labels may be disposed on and/or adjacent to the windows of the sensor and indicator. For example, the labels may be disposed on the windows, such that when the reservoirs associated with the windows of the sensor and indicator are vacant, the labels may be illegible, and when the reservoirs contain the fluid, the labels may be legible. The manner by which fluid may travel from the reservoir associated with user-controlled activator 404 and to each of the reservoirs of temperature sensor 406 and indicator 408 will be described in greater detail below with respect to FIGS. 4B-4E, in accordance with some embodiments.

FIG. 4B shows a back-end of detector 400 comprising a fluid reservoir 428 and one or more substrate pathways 430, 432. As described above, fluid may be configured to travel from fluid reservoir 428 associated with user-controlled activator 404 upon activation of detector 400. In some embodiments, fluid reservoir 428 may be fluidly connected to the reservoir of temperature sensor 406 via one or more capillaries (e.g., fluid pathways). In some embodiments, the interface between reservoir 428 and a capillary configured to connect reservoir 428 to the reservoir of temperature sensor 406 may comprise one or more valves (not illustrated). For example, by engaging with user-controlled activator 404 and therefore inducing pressure to reservoir 428, a valve at the interface may be actuated to allow fluid contained in reservoir 428 to travel through one or more capillaries towards the reservoir of temperature sensor 406. In some embodiments, the valve at the interface may be actuated upon detection of a temperature change (e.g., breach temperature reached or exceeded) by temperature sensor 406. In some embodiments, the time required to travel from reservoir 428 to temperature sensor 406 may be negligible and independent of the temperature of the fluid.

In some embodiments, the fluid may be contained within the reservoir of temperature sensor 406 until one or more breach temperatures of the fluid are detected. In some embodiments, the fluid in detector 400 may be selected such that if a first breach temperature (e.g., 10° C., 12° C., 14° C., etc.) of the fluid is detected, at least a portion of the fluid may begin to travel through a first substrate pathway 430 (e.g., membrane, gel, etc.). For example, upon detection of the first breach temperature, a valve between the reservoir of the temperature sensor 406 and the substrate pathway 430 may be actuated such that fluid may travel into substrate pathway 430. In some embodiments, if a second breach temperature (e.g., 16° C., 18° C., 20° C., etc.) of the fluid is detected, at least a portion of the fluid may begin to travel through a second substrate pathway 432. For example, upon detection of the second breach temperature, a valve between the reservoir of the temperature sensor 406 and the substrate pathway 432 may be actuated such that fluid may travel into substrate pathway 432. In some embodiments, each of the first and second substrate pathway 430, 432 may be configured such that the fluid may travel through the pathways for specified amounts of time. For example, the fluid may travel through the first substrate pathway 430 for a first specified amount of time (e.g., 50 minutes), and the second substrate pathway 432 for a second specified amount of time (e.g., 10 minutes). Thus, if the second (e.g., higher) breach temperature is detected, the fluid may begin to travel through the second substrate pathway 432, which may require less time to reach the indicator 408.

In some embodiments, detector 400 may comprise a plurality of substrate pathways configured to correspond with various durations of time in which the pharmaceutical product is ready to be used. For example, in addition to or in place of one or more of the substrate pathways described above, detector 400 may comprise a third substrate pathway, wherein the fluid may travel through the third pathway and to indicator 408 for a specified amount of time regardless of detected temperatures.

The substrate pathway may comprise a porous membrane, gel, or other material configured to cause fluid to travel through the pathway for a specified amount of time. In some embodiments, the size (e.g., length, width, etc.) of the substrate pathway in detector 400 may be configured to cause the fluid to travel for a specified amount of time. The specified amount of time may correspond with one or more requirements for use of the pharmaceutical product. For example, a given API contained within the pharmaceutical product may be required to rest at an ambient (e.g., room) temperature for a specific amount of time, and/or may be required to reach an appropriate use temperature (e.g., desired temperature) before using the pharmaceutical product. Thus, the one or more substrate pathways 430, 432 may be configured to correspond with the required amount of time, or, in some embodiments, may be configured such that when the fluid reaches the indicator 408, the API will be at the desired temperature for use.

In some embodiments, the fluid may be contained within the reservoir of temperature sensor 406 for a negligible amount of time prior to traveling into one or more substrate pathways 430, 432. For example, upon the fluid reservoir of temperature sensor 406 reaching a threshold pressure, one or more valves at the interface between temperature sensor 406 and one or more substrate pathways 430, 432 may be actuated to allow fluid to travel into the pathways. In some embodiments, as described above, the fluid may travel through the one or more substrate pathways 430, 432 for a specified amount of time. In some embodiments, the specified amount of time may be independent of the temperature of the fluid.

In some embodiments, the fluid may be contained within the reservoir of temperature sensor 406 until a breach temperature is reached or exceeded. Upon the breach temperature being reached, the fluid of detector 400 may travel (e.g., via one or more capillaries, etc.) into indicator 408. For example, the breach temperature may indicate that the pharmaceutical product is ready to be used, thus the fluid may travel from the temperature sensor 406 to the indicator 408 for a negligible amount of time (e.g., the fluid pathway fluidly connecting the indicators may not function as a timing mechanism).

In some embodiments, the one or more substrate pathways 430, 432 may be fluidly connected to an indicator 408. As described above, the indicator 408 may be configured to indicate if the pharmaceutical product is ready to be used. Thus, when the fluid of detector 400 travels through the one or more substrate pathways 430, 432 it may travel into a reservoir of the indicator 408. In some embodiments, indicator 408 may indicate when the pharmaceutical product is ready to be used once fluid traveling from the first substrate pathway 430 or the second substrate pathway 432 reaches the reservoir of indicator 408 (e.g., whichever occurs first).

FIG. 4C shows a fluid-based detector 400 comprising more than one fluid. In some embodiments, the fluid of reservoir 434 may be different from the fluid of reservoir 428 such that the two individual fluids are configured to travel to temperature sensor 406, for example, upon different breach temperatures being reached or exceeded. For example, upon activation via user-controlled activator 404 (described in greater detail above) a first fluid (e.g., fluid in reservoir 428) may be configured to travel to temperature sensor 406 if a first breach temperature (e.g., 10° C., 12° C., 14° C., etc.) is reached. Likewise, upon activation via user-controlled activator 404, a second fluid (e.g., fluid in reservoir 434) may be configured to travel to temperature sensor 406 if a second breach temperature (16° C., 18° C., 20° C., etc.) is reached (e.g., whichever occurs first). In some embodiments, the fluid in reservoir 428 may travel at a first rate and the fluid in reservoir 434 may travel at a second rate, the rates dependent on the detected temperature. The fluid from reservoirs 428, 434 may travel from temperature sensor 406 to indicator 408 to indicate when the pharmaceutical product is ready to be used in a similar manner as described above with respect to FIG. 4B.

In some embodiments, the substrate pathways of detector 400 may be embodied in a variety of configurations. The configurations of each of the substrate pathways may be selected to correspond with specified amounts of time, such that the time required to travel through a given substrate pathway corresponds with the amount of time required for the pharmaceutical product to be ready to be used. FIG. 4D shows a detector 400 comprising one or more gel-filled fluid pathways 436, 438 configured to transport the fluid of the detector from temperature sensor 406 to indicator 408 to indicate if the pharmaceutical product is ready to be used. In some embodiments, one or more properties of the fluid pathways 436, 438 may be varied to correspond with the amount of time required for a pharmaceutical product to reach a desired temperature. In some embodiments, the properties of the fluid pathways 436, 438 may be varied to correspond with the amount of time the pharmaceutical product must rest outside of a controlled cooling environment prior to use. For example, the length, width, configuration, and type of gel within the pathways 436, 438 may be manipulated to correspond with one or more specified amounts of time. In some embodiments, the interface between temperature sensor 406 and the one or more pathways 436, 438 may comprise one or more valves, such that the one or more valves may be actuated upon one or more breach temperatures being reached or exceeded to allow fluid to travel to indicator 408. In some embodiments, rather than requiring a breach temperature to be reached or exceeded, fluid may be configured to travel from temperature sensor 406 and into the one or more gel-filled pathways 436, 438 upon a threshold amount of pressure being reached due to fluid traveling from the fluid reservoir 428 to temperature sensor 406 after activation (e.g., via user-controlled activator 404). In some embodiments, detector 400 may comprise one or more exhaust paths 440. The exhaust path may be configured to aid fluid circulation in detector 400 by providing a path in which remaining gel of the pathways that does not interact with the fluid of detector 400 may enter as fluid travels through the one or more gel-filled fluid pathways towards indicator 408. As shown in FIG. 4D, the exhaust path may fluidly connect the reservoir of indicator 408 to the reservoir 428 associated with user-controlled activator 404, for example.

In some embodiments, detector 400 may comprise more than one fluid configured to interact and indicate if a pharmaceutical product is ready to be used. For example, FIG. 4E illustrates a detector 400 comprising a gas and a fluid (e.g., liquid, gel, etc.). In some embodiments, the gas may be contained within the reservoir 428 associated with user-controlled activator 404 such that upon activation, the gas may (e.g., due to induced pressure in reservoir 404) travel from reservoir 428 and through one or more capillaries towards temperature sensor 406. In some embodiments, the one or more capillaries fluidly connecting reservoir 428 and a reservoir of temperature sensor 406 may comprise the fluid (e.g., liquid, gel, etc.), such that upon activation of detector 400, the gas may cause the fluid to travel from the capillary and into temperature sensor 406 to indicate that detector 400 is active. In some embodiments, temperature sensor 406 may be fluidly connected to the indicator 408 via one or more substrate pathways 430 (e.g., capillaries). The fluid of detector 400 may travel from temperature sensor 406 to indicator 408 for a specified amount of time configured to, for example, correspond with the amount of time required to rest prior to use of the pharmaceutical product. In some embodiments, the specified amount of time my be configured to correspond with the amount of time required for the pharmaceutical product to reach an appropriate use temperature.

In some embodiments, detector 400 may comprise one or more valves, for example at the interface between reservoir 428 and the capillary fluidly connecting the reservoir to temperature sensor 406. Thus, upon a user activating detector 400, the gas contained within the reservoir associated with user-controlled activator 404 may be configured to actuate the valve to allow gas to travel into the capillary. In some embodiments, the interface between the capillary and temperature sensor 406 may instead or additionally comprise a valve configured to be actuated upon gas entering the capillary and increasing the pressure in the capillary, thus causing fluid in the capillary to enter through the valve into the reservoir of temperature sensor 406. In some embodiments, the one or more valves may be actuated upon one or more breach temperatures of the fluid being detected. In some embodiments, fluid may begin to travel from temperature sensor 406 and towards indicator 408 upon a breach temperature being reached or exceeded. In some embodiments, detector 400 may comprise one or more air vents configured to allow any trapped gas of the detector 400 to exit. For example, air vent 440 may be fluidly connected to the reservoir of indicator 408 such that as fluid (e.g., liquid, gel, etc.) travels through one or more reservoirs of detector 400 towards the reservoir of indicator 408, gas may be released from air vent 440.

### Liquid Crystal Sensors for Pharmaceutical Products

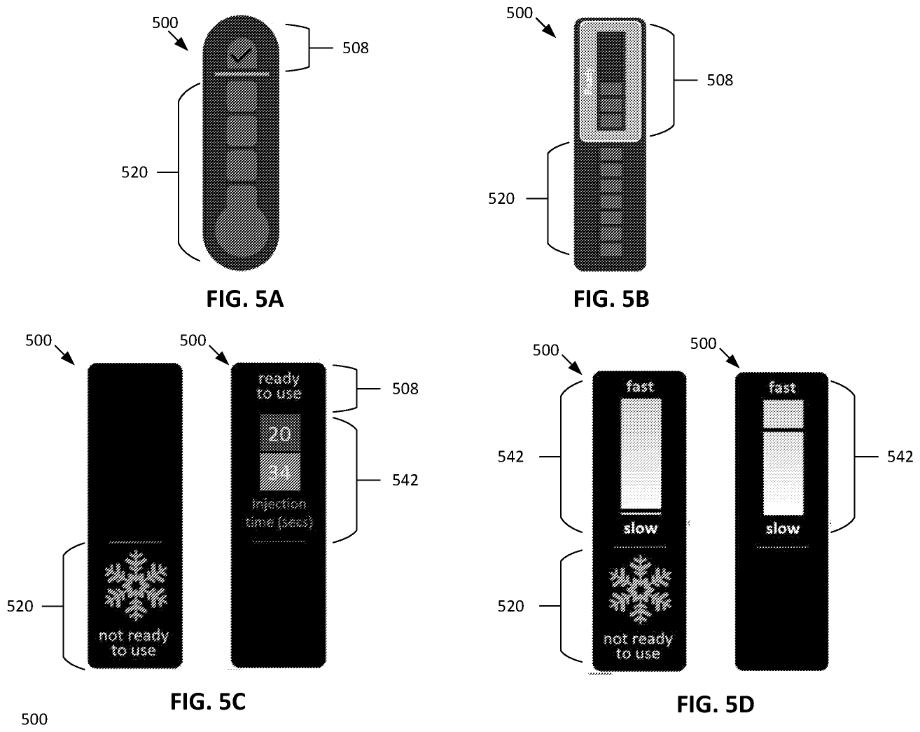

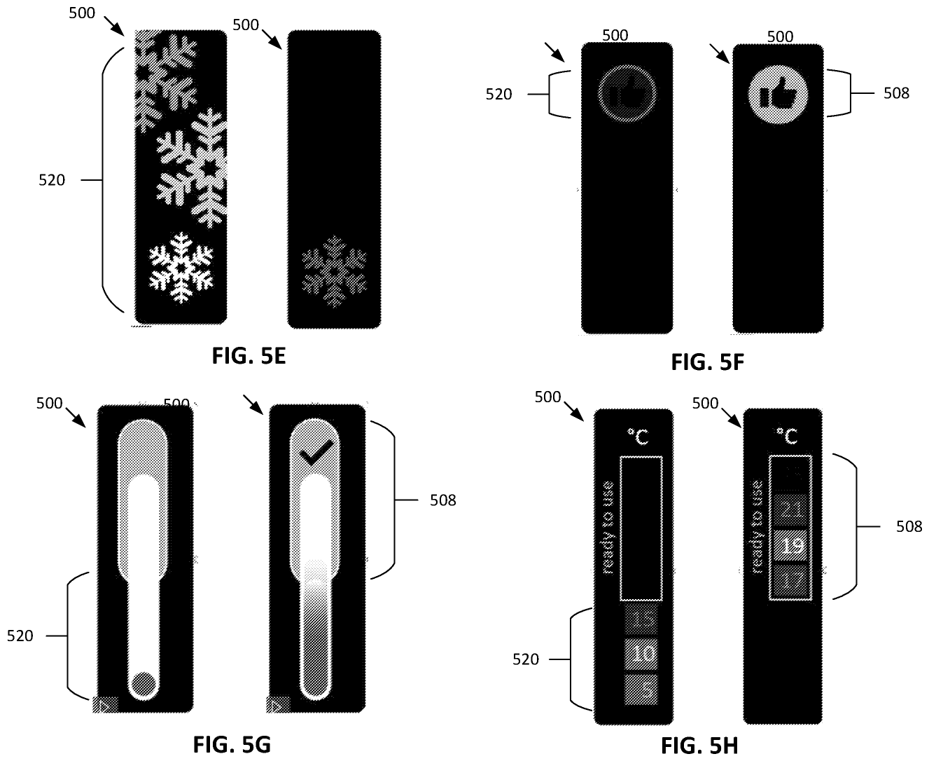

In some embodiments, detector 100 described above with respect to FIG. 1 may be embodied in a liquid crystal (e.g., thermochromic) sensor that includes liquid crystals that may detect a change in temperature of the pharmaceutical product and change color to indicate if the pharmaceutical product is ready to be used. FIGS. 5A-5H show various liquid crystal sensors configured to detect the temperature of and indicate if a pharmaceutical product is ready to be used, according to some embodiments.

FIGS. 5A-5H shows a liquid crystal sensor 500 configured to detect a change in temperature of a pharmaceutical product and indicate when the pharmaceutical product is ready to be used. For example, the liquid crystals disposed on a polymer may be configured to change color at specified temperatures and/or times to indicate if the pharmaceutical product is ready to be used. In some embodiments, liquid crystal sensor 500 may comprise any one or more features of detectors 100, 200, and/or 400 described above with respect to FIG. 1, FIG. 2A-2F, and/or FIG. 4A-4E.

As shown in FIG. 5A, liquid crystal sensor 500 may comprise a first indicator 520 configured to indicate if the pharmaceutical product is not ready to be used, and a second indicator 508 configured to indicate if the pharmaceutical product is ready to be used. In some embodiments, one or more of the first and second indicators may comprise liquid crystals configured to detect temperatures of the pharmaceutical product and change colors as the temperature of the pharmaceutical product increases (e.g., warms). In some embodiments, the liquid crystals of liquid crystal sensor 500 may be configured such that they resemble a meter upon changing color, as shown in FIG. 5A. Indicator 508 may be configured to correspond with a portion of the meter that may illustrate if the pharmaceutical product is ready to be used. For example, indicator 508 may comprise a colored marking that may separate a first portion of the meter (e.g., when the pharmaceutical product is not ready to be used) from a second portion of the meter (e.g., when the pharmaceutical product is ready to be used). In some embodiments, indicator 508 may additionally or instead comprise an icon (e.g., check mark, thumbs-up, text labels, etc.) disposed adjacent to and/or within the second portion of the meter to indicate if the pharmaceutical product is ready to be used.

In some embodiments, the liquid crystals may be configured to first change color at an end of the meter within the first indicator 520 upon removing the pharmaceutical product with the liquid crystal sensor 500 attached thereon from a controlled cooling environment (e.g., refrigerator, freezer, etc.). As the pharmaceutical product continues to warm to an appropriate use temperature (e.g., ambient temperature), additional liquid crystals of sensor 500 may change color to “fill” the meter. For example, liquid crystals between the end of the meter within indicator 520 and the second indicator region 508 may change color over time as the detected temperature increases. After a specified passage of time and/or if an ambient temperature is detected, the liquid crystals associated with the second indicator 508 may be configured to change color to indicate that the pharmaceutical product is ready to be used.

FIG. 5B shows a liquid crystal sensor 500 comprising a first indicator 520 configured to indicate if the pharmaceutical product is not ready to be used, and a second indicator 508 configured to indicate if the pharmaceutical product is ready to be used. Like sensor 500 described above with respect to FIG. 5A, one or more of the indicators may comprise liquid crystals. For example, liquid crystals within indicator 520 may be configured to “fill” a meter visualization as the detected temperature of the pharmaceutical product warms. The liquid crystals may be configured to change color in a successive pattern such that the meter fills over time in a direction towards second indicator 508. In some embodiments, second indicator 508 may comprise a colored box configured to denote a portion of the meter that may correspond to when the pharmaceutical product is ready to be used. For example, when liquid crystals disposed within the box of indicator 508 change color, the pharmaceutical product may be ready to be used. In some embodiments, the box of indicator 508 may be permanently visible (e.g., may not comprise liquid crystals configured to change color based on detected temperatures). In some embodiments, indicator 508 may be emphasized in a different color from the color of the liquid crystals (e.g., the liquid crystals illustrating the meter may be red, and the indicator 508 may be green). In some embodiments, indicator 508 may comprise a label configured to inform the user that the pharmaceutical product is ready to be used when the liquid crystals disposed within the indicator 508 change color (e.g., “ready,” “ready to use,” etc.).

In some embodiments, liquid crystal sensors may additionally provide one or more expected injection times, a detected temperature of the pharmaceutical product, a time in which the pharmaceutical product will be ready to be used, etc. For example, liquid crystal sensor 500 shown in FIG. 5C may indicate with one or more of an icon and/or text fields when the pharmaceutical product is not ready to be used, and after a passage of time, may indicate that the pharmaceutical product is ready to be used (508) in addition to one or more injection times 542. For example, a first injection time may correspond to the amount of time for injection of the API if it is injected at a cool temperature (e.g., high viscosity) prior to the pharmaceutical product reaching an appropriate temperature for use. A second displayed injection time may correspond to the amount of time for injection of the API if it is injected at the appropriate temperature for use (e.g., the injection time may be estimated and displayed in advance of the time to inject the API).

Likewise, FIG. 5D shows a liquid crystal sensor 500 comprising an indication (e.g., an icon and/or text field) 520 when the pharmaceutical product is not ready to be used, in addition to a meter configured to dynamically update to display the injection speed (542). In some embodiments, the meter may be consistently displayed on liquid crystal sensor 500, and an indicator on the meter may be configured to move (e.g., liquid crystals may change color) based on the detected temperature. For example, the indicator may move from the “slow” end of the meter to the “fast” end of the meter as the pharmaceutical product warms to indicate that the injection time (e.g., rate) is increasing. As shown in FIG. 5D, when the pharmaceutical product is ready to be used, liquid crystals previously indicating the status of the pharmaceutical product (520) may be configured to change color, and liquid crystal sensor 500 may display the expected rate of injection of the API.

FIG. 5E shows a liquid crystal sensor 500 comprising a plurality of indicators (e.g., graphics, icons, etc.) configured to indicate if a pharmaceutical product is ready to be used. For example, as liquid crystal sensor 500 detects a change in temperature of the pharmaceutical product, the liquid crystals comprising the indicator may change color (e.g., fade) over time, until the detector is void of indicators. Upon the disappearance of indicator 520, the pharmaceutical product may be ready to use.

FIG. 5F shows a liquid crystal sensor 500 comprising an indicator 520 configured to indicate that the pharmaceutical product is not ready to be used. For example, indicator 520 may comprise an empty icon (e.g., an outline of a shape, such as a circle, square, oval, star, triangle, etc.). As the liquid crystals detect a change in temperature, the empty icon may be configured to change colors and/or fill, as illustrated by indicator 508. For example, the indicator may change from a first color (e.g., red) to a second color (e.g., green). The indicator 508 configured to indicate that the pharmaceutical product is ready to be used may additionally comprise one or more ubiquitous icons (e.g., a thumbs-up, star, check mark, etc.) that may appear when the pharmaceutical product is ready to be used.

FIG. 5G shows a liquid crystal sensor comprising one or more features of FIGS. 5A-5B above. For example, liquid crystal sensor 500 illustrated in FIG. 5G may comprise a meter with a first portion/end configured to indicate when the pharmaceutical product is not ready to be used (520) and a second portion/end configured to indicate when the pharmaceutical product is ready to be used (508). In some embodiments, the liquid crystals comprising the meter may dynamically change color over time as the temperature detected increases, from the first end to the second end to indicate when the pharmaceutical will be ready and/or is ready to be used.

FIG. 5H shows a liquid crystal sensor configured to indicate when the pharmaceutical product is ready to be used and relay information regarding the temperature of the pharmaceutical product as the product warms. For example, liquid crystal sensor 500 may comprise a scale with temperature benchmarks indicated therein, such that at a first end of the scale (520), cool temperatures may be displayed as they are detected. After a passage of time, temperature benchmarks on a second end of the scale (e.g., corresponding to when the pharmaceutical product is ready to be used) may be gradually displayed. In some embodiments, the appropriate temperatures for use may be emphasized (e.g., outlined with an indicator 520) to indicate when the pharmaceutical product is ready to be used.

Each of the liquid crystal sensors 500 described above with respect to FIGS. 5A-5H may comprise one or more features described with respect to another embodiment. For example, detector 500 described with respect to FIG. 5A may comprise one or more features of detector 500 described with respect to FIG. 5H (e.g., detector 500 of FIG. 5A may additionally display temperature information). The various embodiments and figures of liquid crystal sensors 500 are not intended to be interpreted as individual embodiments, but rather to demonstrate a variety of features that may be implemented in combination using liquid crystal (e.g., thermochromic) sensing to indicate at least when the pharmaceutical product is ready to be used.

### Color Changing Materials for Pharmaceutical Products

In some embodiments, one or more components of detector 100 described above with respect to FIG. 1 may be embodied within the pharmaceutical product itself. For example, the container of the pharmaceutical product may comprise one or more color-changing materials configured to detect the temperature of the pharmaceutical product and change color when the pharmaceutical product is at an appropriate temperature for use. In some embodiments, the color-changing materials of the pharmaceutical product may be configured to change color over a specified duration of time, such that the pharmaceutical product is ready to be used upon the specified amount of time elapsing. In some embodiments, the rate at which the color-changing material of the pharmaceutical product changes color may be selected based on one or more properties (e.g., type, volume, etc.) of the API contained within the pharmaceutical product.

In some embodiments, the container of the pharmaceutical product may comprise a window (e.g., on a syringe holder) configured to change color at an appropriate use temperature. In some embodiments, the window may additionally or instead change color after a specified amount of time has elapsed after the pharmaceutical product has been removed from the fridge. In some embodiments, the window may change from opaque to transparent, or vice versa. In some embodiments, an additional or different component of the pharmaceutical product may be configured to change color upon detection of an appropriate use temperature and/or passage of a specified amount of time. For example, a cap of an autoinjector may be configured to change from opaque to transparent (or vice versa) if the pharmaceutical product is ready to be used.

In some embodiments, one or more components of the pharmaceutical product may comprise a color-changing polymer. For example, in the instance the container of the pharmaceutical product is a drug delivery device (e.g., injector), a needle guard of the device may be configured to change color if the pharmaceutical product is ready to be used (e.g., based one or more of a temperature and/or time being reached). In some embodiments, a plunger of the drug delivery device may additionally or instead be configured to change color if the pharmaceutical product is ready to be used. In some embodiments, one or more internal components of the drug delivery device may change color when the pharmaceutical product is ready to be used, and a user may view the internal color-changing component through a window (e.g., on the syringe holder).

In some embodiments, a pharmaceutical product may comprise any combination of detection systems described above. For example, the pharmaceutical product may comprise a liquid crystal sensor configured to display injection time, and one or more color-changing materials configured to indicate when the pharmaceutical product is ready to be used. In some embodiments, a pharmaceutical product may comprise one or more illuminators (e.g., LED light indicators), and an LED display screen configured to display an injection time. The provided examples are not intended to be limiting, and it is to be understood that any combination of features of the detection systems described herein may be combined at least to indicate when the pharmaceutical product is ready to be used.

Method of Indicating that a Pharmaceutical Product is Ready to be Used

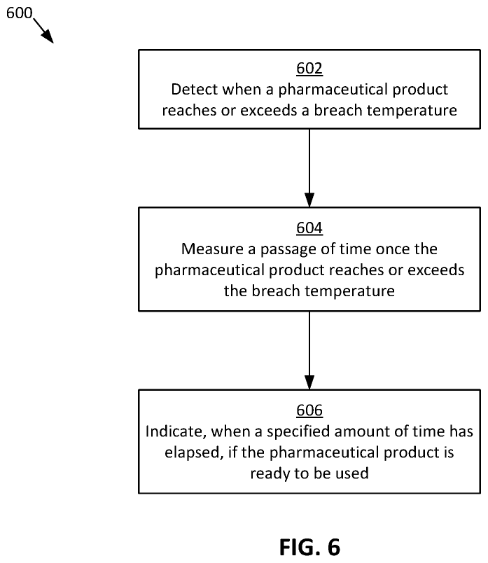

As described above, the detectors disclosed herein may be configured to be attached to a pharmaceutical product and may detect temperatures of and indicate when a pharmaceutical product is ready to be used. The pharmaceutical product may comprise a container configured to contain an active pharmaceutical ingredient (API). FIG. 6 shows a method 600 for detecting temperatures of and indicating when a pharmaceutical product is ready to be used, according to some embodiments.

At step 602, a temperature sensor of the detector may detect temperatures of the pharmaceutical product. At step 604, at least one timer of the detector may begin to measure a passage of time if a detected temperature of the pharmaceutical product reaches or exceeds a breach temperature. In some embodiments, as described above, the timer may be embodied in a processor of the detector. At step 606, at least one indicator of the detector may generate an indication that the pharmaceutical product is ready to be used if a specified amount of time has elapsed on the at least one timer. In some embodiments, the specified amount of time may be selected to correspond to when the API contained within the pharmaceutical product will be ready to be used. In some embodiments, the specified amount of time may be selected to correspond to an amount of time in which the API contained within the pharmaceutical product will, when disposed in an environment that is within a standard room temperature range, reach a desired temperature.

The foregoing description sets forth exemplary systems, methods, techniques, parameters, and the like. It should be recognized, however, that such description is not intended as a limitation on the scope of the present disclosure but is instead provided as a description of exemplary embodiments.

Although the description herein uses terms first, second, etc. to describe various elements, these elements should not be limited by the terms. These terms are only used to distinguish one element from another.

1. A detector configured to be attached to a pharmaceutical product that contains an active pharmaceutical ingredient (API) therein, the detector comprising:
a temperature sensor configured to detect a temperature of the pharmaceutical product;
at least one timer configured to start measuring a passage of time if the temperature of the pharmaceutical product detected by the temperature sensor reaches or exceeds a breach temperature; and
at least one indicator configured to generate, if a specified amount of time has elapsed on the at least one timer, an indication that the pharmaceutical product is ready to be used.

2. (canceled)

3. The detector of claim 1, wherein the specified amount of time is selected to correspond to an amount of time in which the active pharmaceutical ingredient (API) contained within the pharmaceutical product will, when disposed in an environment that is within a standard room temperature range, reach a desired temperature.

4. (canceled)

5. A pharmaceutical product, comprising:
a container configured to contain an active pharmaceutical ingredient (API); and
the detector of claim 1, wherein the detector is disposed adjacent to the container.

6. The pharmaceutical product of claim 5, wherein the container comprises an injector configured to contain the active pharmaceutical ingredient (API).

7. (canceled)

8. The pharmaceutical product of claim 5, wherein the temperature sensor determines a temperature of the active pharmaceutical ingredient (API) based at least in part on a temperature of the container.

9. (canceled)

10. The detector of claim 1, comprising a user-controlled activator configured to activate the temperature sensor.

11. The detector of claim 1, wherein the at least one timer is configured to start measuring a second passage of time if the temperature of the pharmaceutical product detected by the temperature sensor reaches or exceeds a second breach temperature.

12. The detector of claim 11, wherein the second breach temperature is greater than the breach temperature.

13. The detector of claim 11, wherein a first timer of the at least one timer is configured to measure the passage of time if the breach temperature is detected, and a second timer of the at least one timer is configured to measure the second passage of time if the second breach temperature is detected.

14. The detector of claim 11, wherein the passage of time measured by the at least one timer is a longer duration of time than the second passage of time measured by the at least one timer.

15. (canceled)

16. (canceled)

17. (canceled)

18. (canceled)

19. (canceled)

20. (canceled)

21. (canceled)

22. (canceled)

23. The detector claim 1, comprising a fluid configured to travel through the temperature sensor and the at least one timer.

24. The detector of claim 23, comprising a user-controlled activator configured to cause, if a user-controlled activator is activated, the fluid to travel from a reservoir associated with the user-controlled activator and to the temperature sensor.

25. (canceled)

26. The detector of claim 23, wherein the fluid is configured to travel, if the pharmaceutical product reaches or exceeds the breach temperature, from the temperature sensor and through the at least one timer.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. A detector configured to be attached to a pharmaceutical product that contains an active pharmaceutical ingredient (API) therein, the detector comprising:
a temperature sensor configured to detect temperatures of the pharmaceutical product;
at least one processor configured to calculate changes between the detected temperatures over time; and
at least one indicator configured to generate, if a change of the calculated changes between the detected temperatures over time meets or falls below a predefined threshold, an indication that the pharmaceutical product is ready to be used.

58. The detector of claim 57, wherein the predefined threshold is selected to correspond to when the active pharmaceutical ingredient (API) within the pharmaceutical product will be ready to be used.

59. The detector of claim 57, wherein calculating changes between the detected temperatures over time comprises determining a difference between a first temperature and a second temperature, the first and second temperature detected a predefined amount of time apart.

60. The detector of claim 59, wherein the at least one processor is configured to generate a ratio of the difference between the first and second temperature over the predefined amount of time apart and compare the ratio to the predefined threshold.

61. (canceled)

62. (canceled)

63. (canceled)

64. (canceled)

65. A liquid crystal sensor configured to be attached to a pharmaceutical product that contains an active pharmaceutical ingredient (API) therein, the liquid crystal sensor comprising:
liquid crystals configured to change color to indicate increasing temperatures of the pharmaceutical product as the pharmaceutical product warms;
at least one indicator configured to indicate when the API within the pharmaceutical product reaches a specified temperature or temperature range, wherein the specified temperature or temperature range is indicative of when the pharmaceutical product is ready to be used.

66. The liquid crystal sensor of claim 65, wherein a portion of the liquid crystals are adjacent to the at least one indicator on the liquid crystal sensor and are configured to change color when the pharmaceutical product is ready to be used.

67. The liquid crystal sensor of claim 65, wherein the liquid crystals comprise a visual meter that indicates the increasing temperatures of the pharmaceutical product as the pharmaceutical product warms.

68. (canceled)

69. The liquid crystal sensor of claim 65, wherein the at least one indicator is configured to indicate an expected duration for the injection.
